(19) United States
(12) Patent Application Publication
(10) Pub. No.: US 2023/0310645 A1
(43) Pub. Date: Oct. 5, 2023

(54) NANOPARTICLE COMPOSITION FOR THE DELIVERY OF NUCLEIC ACID AGENTS

(71) Applicant: Tiba Biotech LLC, Cambridge, MA (US)

(72) Inventors: Poulami Talukder, Woburn, MA (US); Maria Varghese, Woburn, MA (US)

(73) Assignee: Tiba Biotech LLC, Cambridge, MA (US)

(21) Appl. No.: 18/130,122

(22) Filed: Apr. 3, 2023

Related U.S. Application Data
(60) Provisional application No. 63/326,541, filed on Apr. 1, 2022.

(51) Int. Cl. A61K 47/69; A61K 31/711; A61K 31/7105

(52) U.S. Cl. CPC A61K 47/6935; A61K 31/711; A61K 31/7105; B82Y 5/00

(57) ABSTRACT

The present disclosure relates to nanoparticle compositions for delivery of nucleic acid agents. The nanoparticle composition disclosed includes a carrier containing a dendritic polymer skeleton, a PEG moiety, an amine, and a hydrophobic unit. The size of the PEG moiety may be limited, potentially reducing or avoiding anti-PEG antibody induction.

### CROSS-REFERENCE TO RELATED APPLICATIONS

This application claims the benefit of priority of U.S. Provisional Patent Application Ser. No. 63/326,541, filed on Apr. 1, 2022, and titled “DENDRITIC-POLYMER CONJUGATES FOR GENE DELIVERY,” which is incorporated by reference herein in its entirety.

### FIELD OF THE INVENTION

The present invention generally relates to the field of nanoparticles. In particular, the present invention is directed to nanoparticle compositions for the delivery of nucleic acid agents.

### BACKGROUND

PEGylated carriers have been widely used in the field of drug delivery and bioconjugation due to their long circulatory half-life, biocompatibility, and low toxicity. It is increasingly reported, however, that treating patients with PEGylated drugs leads to the formation of antibodies that specifically recognize PEG (i.e., anti-PEG antibodies). Consequently, treating patients who have acquired anti-PEG antibodies with existing PEGylated drugs may result in accelerated blood clearance (ABC) with enhanced accumulation of PEG-conjugates in the liver and spleen, low drug efficacy, hypersensitivity, and potentially life-threatening side effects.

### SUMMARY OF THE DISCLOSURE

In an aspect, nanoparticle composition for delivery of a nucleic acid agent comprises: a nucleic acid agent and a carrier, wherein the carrier comprises: a dendritic-polymer skeleton; an amine; and a hydrophobic unit, wherein the dendritic-polymer skeleton comprises a PEG moiety, and wherein the amine connects the hydrophobic unit to the dendritic-polymer skeleton.

In another aspect, a method of managing disease comprises: administering a therapeutically effective amount of a nanoparticle composition to a subject, wherein the nanoparticle composition comprises: a nucleic acid agent and a nucleic acid carrier, wherein the carrier comprises: a dendritic-polymer skeleton; an amine; and a hydrophobic unit, wherein the dendritic-polymer skeleton comprises a PEG moiety, wherein the amine connects the hydrophobic unit to the dendritic-polymer skeleton.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic diagram illustrating an exemplary embodiment of a nanoparticle composition for delivery of nucleic acid agents;

FIGS. 2A-2O are diagrams illustrating exemplary embodiments of formulas of carriers which may be used in nanoparticle compositions for delivery of nucleic acids where A is an amine, B is a hydrophobic unit, m is 0 to 10, Y can be selected from alkyne, azide, amino (NH2), NHMe, NHBoc, Br, I, aminooxy, aryl, carboxylic acid (CO2H), sulfonic Acid, NHS ester, methyl, hydroxy, phosphonate, silane, tosylate, X-PEG where X is NH, NMe, NBoc; P can be selected from PEG derivatives and PEG alternatives;

FIG. 3A-3B illustrate an exemplary embodiment of a synthetic scheme of an asymmetrical PEGylated dendrimer;

FIG. 4 is an illustration of an exemplary embodiment of a synthetic route of a PEGylated dendron where focal point of the dendron is modified with poly(ethylene glycol);

FIG. 5A-5B is a plot demonstrating an experimental result showing a distribution of nanoparticle composition measured as an intensity (Z average) based on size of the nanoparticles in an embodiment;

FIG. 6 is an illustration of an agarose gel showing binding of a PEGylated-dendron with RNA;

FIG. 7A-7C are graphical diagrams illustrating exemplary embodiments of quantification in vitro (7A-7B) and in vivo (7C) of secreted alkaline phosphatase (SEAP) expression after administration of nanoparticle formulations based on PEGylated dendron and PEGylated dendrimer in an exemplary embodiment;

FIG. 8 is a flow diagram illustrating an exemplary embodiment of a method for managing a disease or condition in a subject;

FIG. 9A-9C illustrates an exemplary embodiment of a synthetic scheme of an asymmetrical PEGylated dendrimer;

FIG. 10 is an illustration of an exemplary embodiment of a synthetic route of a Polyglycerol dendron conjugate;

FIG. 11A-11B demonstrating a distribution of nanoparticle composition measured as an intensity (Z average) based on size of the nanoparticles in an embodiment;

FIG. 12 is a photograph of an agarose gel showing binding of the PEGylated-dendrimer with RNA in an exemplary embodiment; and

FIG. 13 illustrates quantification in vitro of secreted alkaline phosphatase (SEAP) expression after administration of nanoparticle formulations based on PEGylated dendrimer in an exemplary embodiment.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, the present disclosure relates to nanoparticle compositions for delivery of nucleic acid agents. The nanoparticle compositions described herein include a “carrier” made up of a dendritic polymer skeleton, a PEG moiety conjugated to the dendritic polymer skeleton, a hydrophobic unit, and an amine that connects the dendritic polymer skeleton to the hydrophobic unit. The nanoparticle composition also includes a “nucleic acid agent” payload.

In some embodiments, a PEG moiety is limited in size. For example, a PEG moiety may have a molecular weight of less than 750 Daltons. As another example, a PEG moiety may have a length of 1 to 15 monomers. Limiting molecular weight may help minimize or avoid anti-PEG antibody induction. A branched PEG structure, also appropriate in certain embodiments of the invention, may impact the threshold mass of immune recognition further.

In some embodiments, the nanoparticle composition includes additional components. In some embodiments, the nanoparticle composition further includes a lipid conjugate. A lipid conjugate may include a PEG-lipid, such as DMG-PEG 2000. In some embodiments, the nanoparticle composition further includes an amphipathic lipid. An amphipathic lipid may include a phospholipid, such as DSPC. In some embodiments, the nanoparticle composition further includes cholesterol or a cholesterol derivative. In an embodiment, a nanoparticle composition comprises a carrier, a nucleic acid agent, a lipid conjugate, an amphipathic lipid, and cholesterol or a derivative thereof.

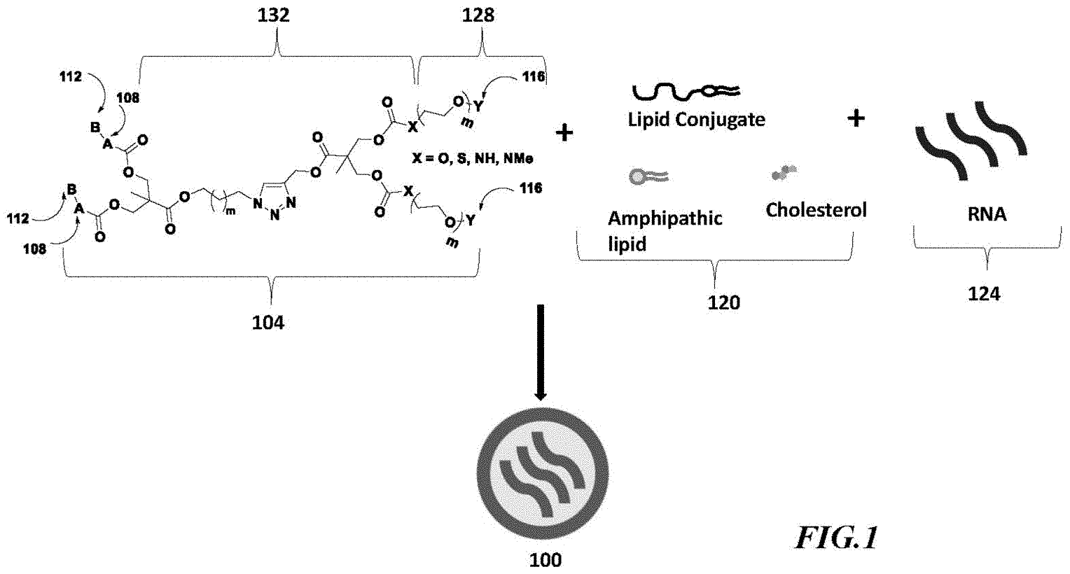

Referring to FIG. 1, an exemplary embodiment of a nanoparticle composition 100 is illustrated. Nanoparticle composition 100 may include one or more nucleic acid agents 124. As used herein, a “nucleic acid agent” is a compound including at least one nucleotide, such as without limitation a DNA or RNA molecule. A nucleic acid agent may be natural or synthetic. A nucleic acid agent may be chemically modified. A nucleic acid agent may have therapeutic or immunogenic properties.

Still referring to FIG. 1, a nucleic acid agent may include ribonucleic acid (“RNA”). Examples of elements of RNA that may be found in nucleic acid agents include without limitation replicon RNA (repRNA), small interfering RNA (siRNA), miRNA, single strand guide RNA (sgRNA), messenger RNA (mRNA), and transfer RNA (tRNA).

Still referring to FIG. 1, replicon RNA (repRNA) is a genome replication-competent, progeny-defective RNA virus genome that is incapable of producing infectious progeny virions. Viral genomes that are typically modified for use as repRNAs include positive strand RNA viruses. Modified viral genomes may function as both mRNA and templates for replication. Small interfering RNA (siRNA) is an RNA (or RNA analog) comprising about 10-50 nucleotides (or nucleotide analogs) which is capable of directing or mediating RNA interference. MicroRNA (miRNA) is small (20-24 nt) regulatory non-coding RNA involved in post-transcriptional regulation of gene expression in eukaryotes by affecting either or both the stability and translation of coding mRNAs. Messenger RNA (mRNA) defines the amino acid sequence of one or more polypeptide chains and is usually single-stranded. mRNA may be translated into protein via protein synthesis. mRNA may be monocistronic or polycistronic mRNA. Monocistronic mRNA contains only one sequence encoding a protein. Polycistronic mRNA contains two or more protein coding sequences.

Still referring to FIG. 1, limiting PEG moiety size is especially important for repRNA vaccine payloads. As repRNA payloads are substantially more immunogenic compared to conventional base-modified mRNA, delivery of these vectors may increase the danger of errantly driving immune responses against residual PEG in a formulation. Therefore, for repRNA-based technologies, reducing the mass of nanoparticle surface-exposed PEG in a vaccine formula would be highly advantageous to circumvent the dangers of anti-material immune responses.

Still referring to FIG. 1, a nucleic acid agent may include a deoxyribonucleic acid (“DNA”). A DNA nucleic acid agent may include, without limitation, a polynucleotide, oligonucleotide, DNA, or cDNA.

Still referring to FIG. 1, a nucleic acid agent may encode a protein, such as a therapeutic protein. A nucleic acid agent may encode an antigen. As used in this disclosure, an “antigen” is defined as a molecule that triggers an immune response. An immune response may involve either antibody production, or the activation of specific immunologically active cells, or both. An antigen may refer to any molecule capable of stimulating an immune response, including macromolecules such as proteins, peptides, or polypeptides. Antigens may include a structural component of a pathogen, a cancer cell, or a derivative thereof. Antigens may be synthesized, produced recombinantly in a host, or may be derived from a biological sample, including but not limited to a tissue sample, cell, or a biological fluid. Example antigens include, without limitation, vaccine antigens, parasite antigens, bacterial antigens, tumor antigens, environmental antigens, therapeutic antigens or allergens. In some embodiments, compositions described herein may be used to create a nucleotide vaccine. As used herein, a “nucleotide vaccine” is a DNA- or RNA-based prophylactic and/or therapeutic composition capable of stimulating an adaptive immune response in the body of a subject by delivering antigen(s). An immune response induced by vaccination may result in development of immunological memory, and the ability of the organism to quickly respond to subsequent encounter with an antigen or infectious agent.

Still referring to FIG. 1, a nucleic acid agent may include a protein-coding mRNA or circular RNA (circRNA), wherein translation of an encoded ORF is driven by cap-dependent or independent mechanisms inside a cell. An encoded protein may exert biological effects intracellularly, or be secreted by a cell to exert autocrine, paracrine, or systemic effects in a subject; a protein produced may serve to replace the activity of an endogenous protein which is either missing from a subject's genome, insufficiently active, or otherwise aberrantly expressed due to a genetic or acquired disorder. Multiple therapeutic genes may be delivered by local or systemic administration to a subject.

Still referring to FIG. 1, a nucleic acid agent may modulate gene expression. A nucleic acid agent may include an antisense oligonucleotide (AON) or a double-stranded small interfering RNA (siRNA). siRNAs may be between 21 and 23 nucleotides in length. An siRNA may include a sequence complementary to a sequence contained in an mRNA transcript of a target gene when expressed within a host cell. An antisense oligonucleotide may be a morpholino antisense oligonucleotide. An antisense oligonucleotide may include a sequence complementary to a sequence contained in an mRNA transcript of a target gene. A nucleic acid agent may include an interfering RNA (iRNA) against a specific target gene within a specific target organism. An iRNA may induce sequence-specific silencing of the expression or translation of a target polynucleotide, thereby down-regulating or preventing gene expression. An iRNA may completely inhibit expression of a target gene. An iRNA may reduce the level of expression of a target gene compared to that of an untreated control. A nucleic acid agent may be a microRNA (miRNA). A miRNA may be a short RNA, e.g., a hairpin RNA (hpRNA). A miRNA may be cleaved into biologically active dsRNA within a target cell by the activity of endogenous cellular enzymes. A nucleic acid agent may include a double-stranded RNA (dsRNA). A dsRNA may be 25 nucleotides in length or longer. A dsRNA may contain a sequence that is complementary to the sequence of a target gene or genes.

Still referring to FIG. 1, a nucleic acid agent may include or may encode an agent that totally or partially reduces, inhibits, interferes with or modulates the activity or synthesis of one or more genes encoding target proteins. For example, a nucleic acid agent may encode an agent that reduces expression of a gene of a host organism. A sequence of a nucleic acid agent may be less than 100% complementary or identical to the nucleic acid sequence of a target gene.

Still referring to FIG. 1, a composition as described herein may be used for targeted, specific alteration of genetic information in a subject. For example, a nanoparticle composition may be administered to a subject, leading to a therapeutic or immunogenic response. As used herein, the term “alteration” refers to any change in the genome in the cells of a subject. An alteration may include, for example, insertion and/or deletion of nucleotides in the sequence of a target gene. An alteration may be a correction of a sequence of a target gene, whereby the sequence is changed to result in a more favorable expression of the gene as manifested by improvements in genotype and/or phenotype of the host organism. Alteration of genetic information may be achieved using genome editing techniques generally known to those skilled in the art, such as CRISPR. An exemplary genome editing system is a Clustered Regularly Interspaced Short Palindromic Repeats (CRISPR) system. In general, “CRISPR system” refers to transcripts and other elements involved in the expression of CRISPR-associated (Cas) genes, including sequences encoding a Cas gene, a tracr (trans-activating CRISPR) sequence, a tracr-mate sequence, a guide sequence, or other sequences and transcripts from a CRISPR locus. One or more tracr mate sequences may be operably linked to a guide sequence before processing or crRNA after processing by a nuclease. tracrRNA and crRNA may be linked and may form a chimeric crRNA-tracrRNA hybrid where a mature crRNA is fused to a partial tracrRNA via a synthetic stem loop to mimic a natural crRNA:tracrRNA duplex. A single fused crRNA-tracrRNA construct is also referred herein as a guide RNA or gRNA, or single-guide RNA (sgRNA). Within an sgRNA, the crRNA portion is identified as the “target sequence” and the tracrRNA is often referred to as the “scaffold.” In an embodiment, the nanoparticle compositions described herein may be used to deliver an sgRNA. The sequence of a target gene may be determined by, for example, illumina dye sequencing.

Still referring to FIG. 1, a nanoparticle composition described herein may be used to apply other exemplary genome editing systems including meganucleases, homing endonucleases, TALEN-based systems, or Zinc Finger Nucleases. A nanoparticle composition may be used to deliver a nucleic acid agent that encodes the sequences for these gene editing tools, and the actual gene products, proteins, or other molecules.

Still referring to FIG. 1, a nanoparticle composition may be used for gene targeting in a subject in vivo or ex vivo, e.g., by isolating cells from the subject, editing genes, and implanting the edited cells back into the subject. An embodiment includes a method comprising administering a nanoparticle composition herein to isolated cells from a subject. A method may include gene targeting. A method may include implanting edited cells back into a subject.

Still referring to FIG. 1, a composition described herein may include more than one nucleic acid agent. For example, a composition may include a mixture of one or more different DNA molecules, RNA molecules, or a combination of the two.

Still referring to FIG. 1, a nucleic acid agent may be non-covalently bound or covalently bound to a carrier. A nucleic acid agent may be bound to a charged carrier through electrostatic interaction. A nucleic acid agent may be bound to a charged carrier through electrostatic interaction and hydrogen bonding.

Still referring to FIG. 1, a nucleic acid agent may be complexed with a carrier. A nucleic acid agent may be encapsulated in a nanoparticle comprising carriers. Such a nanoparticle may also include one or more of: a lipid conjugate, an amphipathic lipid, and cholesterol or a cholesterol derivative, as described below. Encapsulation of a nucleic acid agent may be partial encapsulation or full encapsulation. In the context of nucleic acid agents, full encapsulation may be determined by a RiboGreen® assay. RiboGreen® is an ultra-sensitive fluorescent nucleic acid stain for quantitating oligonucleotides and single-stranded DNA or RNA in solution (available from Thermo Fisher Scientific-US, catalog number R11490).

Referring to FIG. 1, a nanoparticle composition 100 for delivery of a nucleic acid agent is illustrated. This nanoparticle composition includes a carrier 104 that comprises a dendritic skeleton 132, a PEG moiety 128, an amine linker 108, and a hydrophobic unit 112.

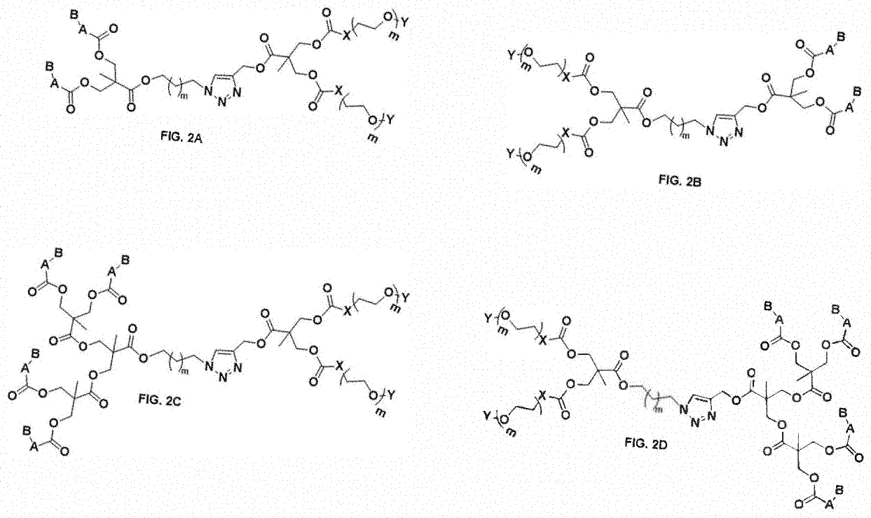

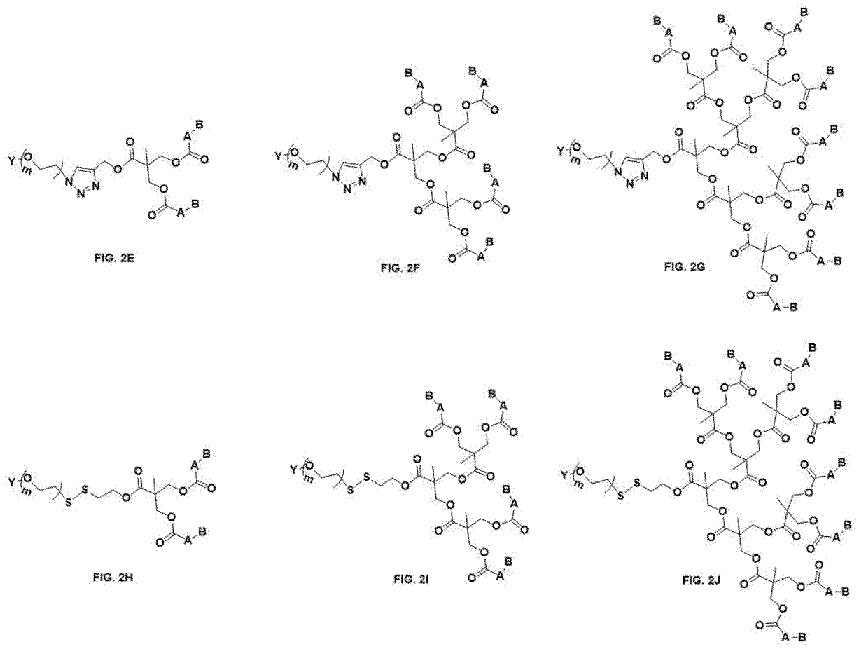

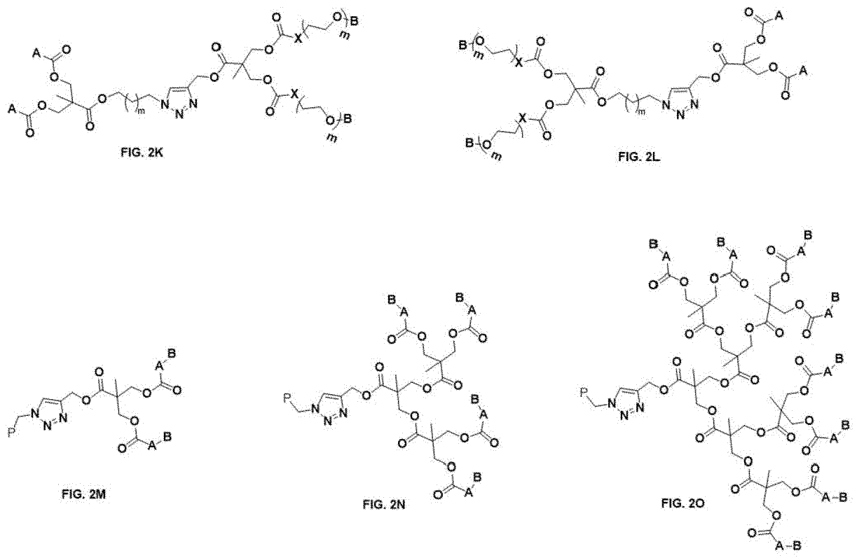

Referring to FIG. 1, a carrier may include a dendritic skeleton 132. A dendritic skeleton may include a dendron. A dendritic skeleton may include a dendrimer. As used herein, a “dendrimer” is a molecular architecture with an interior core and layers (or “generations”) of repeating units which are attached to and extend from this interior core, each layer having one or more branching points, and an exterior surface of terminal groups attached to the outermost generation. Dendrimers have regular dendrimeric or “starburst” molecular structures. A dendritic skeleton may comprise 2,2-bis(hydroxymethyl) propionic acid or 2,2-Bis(hydroxymethyl)butyric acid as the monomeric polyester unit. A dendritic skeleton may be conjugated to a PEG moiety and an amine. FIG. 2A-2L depict various examples of dendritic polymer skeletons in the context of a carrier.

Referring to FIG. 1, a carrier may include an amine. In some embodiments, an amine may include an amine linker 108. As used herein, an “amine linker” is an amine-containing linker that links or connects hydrophobic tails with terminal chemical groups present on a dendrimer or dendron surface. Amines present in an amine linker may include functional groups that contain a basic nitrogen atom with a lone pair. Amines are formally derivatives of ammonia, wherein one or more hydrogen atoms have been replaced by a substituent such as an alkyl group.

Still referring to FIG. 1, amine linker 108 may include a moiety that imparts proton-accepting functionality to a carrier by containing one or more nitrogen atoms with lone pairs. Amine linker may thus be able to accept a free proton (H+) under acidic conditions. A nitrogen atom in an amine linker may be present in the form of a secondary amine. A nitrogen atom in an amine linker may be present in the form of a tertiary amine. An amine linker may be derived, without limitation, from N1-(2-aminoethyl)ethane-1,2-diamine, N1-(2-aminoethyl)propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-aminoethyl)ethane-1,2-diamine), N1-(2-(4-(2-aminoethyl)piperazin-1-yl)ethyl)ethane-1,2-diamine,N1-(2-aminoethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl)(methyl)amino)propan-1-ol, 3,3′-(methylazanediyl)bis(propan-1-ol), N1-(3-aminopropyl)-N1-methylbutane-1,4-diamine, 4-((3-aminopropyl)(methyl)amino)butan-1-ol, 4-((3-hydroxypropyl)(methyl)amino)butan-1-ol, 4-((3-hydroxypropyl)(methyl)amino)butan-1-ol, N1-(4-aminobutyl)-N1-methylbutane-1,4-diamine, 4-((4-aminobutyl)(methyl)amino)butan-1-ol, 4,4′-(methylazanediyl)bis(butan-1-ol), 3-((3-aminopropyl)(ethyl)amino)propan-1-ol, 3,3′-(ethylazanediyl)bis(propan-1-ol), N1-(3-aminopropyl)-N1-ethylbutane-1,4-diamine, 4-((3-aminopropyl)(ethyl)amino)butan-1-ol, 4-(ethyl(3-hydroxypropyl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl)bis(butan-1-ol), 3-((3-aminopropyl)amino)propan-1-ol, N1-(3-aminopropyl)butane-1,4-diamine, 4-((3-hydroxypropyl)amino)butan-1-ol, N1-(4-aminobutyl)butane-1,4-diamine, 3,3′-azanediylbis(propan-1-ol), 4-((3-aminopropyl)amino)butan-1-ol, 4,4′-azanediylbis(butan-1-ol), or N1,N1′-(butane-1,4-diyl)bis(propane-1,3-diamine).

Still referring to FIG. 1, a carrier in nanoparticle composition 100 may include a hydrophobic unit 112. As used herein, a “hydrophobic unit” is a group formed exclusively by hydrophobic atoms and not surrounded by water molecules. Hydrophobic unit 112 may include a C₁-C₂₈ alkyl group. Hydrophobic unit 112 may include a C₁-C₂₀ alkyl group. Hydrophobic unit 112 may include a C₂-C₂₈ alkenyl group. Chain length may be used to control the hydrophobicity and self-assembly properties of a carrier. These groups may optionally be substituted with one to four substituents selected from halogen, —CN, —NO₂, —N₃, C₁-C₆ alkyl, halo(C₁-C₆ alkyl), —OR, —NR₂, —CO₂R, —OC(O)R, —CON(R)₂, —OC(O)N(R)₂, —NHC(O)N(R)₂, —NHC(NH)N(R)₂, C₃-C₈ cycloalkyl, C₃-C₈ cycloalkenyl, aryl, heteroaryl, or heterocycle. Each R may independently be selected from hydrogen, C₁-C₆ alkyl, halo(C₁-C₆ alkyl), C₃-C₈ cycloalkyl, C₃-C₈ cycloalkenyl, aryl, heteroaryl, or heterocycle. Each cycloalkyl, cycloalkenyl, aryl, heteroaryl, and heterocycle may be further optionally substituted with R′, wherein R′ may independently be selected from halogen, —CN, —NO₂, —N₃, C₁-C₆ alkyl, or halo(C₁-C₆ alkyl). A hydrophobic unit may be added by contacting a carrier with a functional reagent such as a fatty acid or its derivatives. A fatty acid may be a saturated or unsaturated fatty acid having C₄-C₂₈ chains. A fatty acid may include, but is not limited to, caprylic acid, capric acid, lauric acid, myristic acid, palmitic acid, stearic acid, oleic acid, linoleic acid, linolenic acid, arachidonic acid, eicosapentanoic acid. A fatty acid derivative may be, but is not limited to, 12-hydroxy-9-cis-octadecenoic acid (ricinoleic acid), 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, or DL-β-hydroxypalmitic acid. Hydrophobic unit 112 may be a methyl, ethyl, propyl, butyl, pentyl, hexyl, heptyl, octyl, decyl, undecyl, dodecyl, tridecyl, tetradecyl, pentadecyl, hexadecyl, heptadecyl, octadecyl, but-3-en-1-yl, oct-7-en-1-yl, 12-tridecenyl, 14-pentadecenyl, 17-octadecenyl, oleyl, linoleyl, arachidoneyl, 16-hydroxyhexadecyl or 12-hydroxy-9-cis-octadecenyl (ricinoleyl) group.

Still referring to FIG. 1, a carrier may include a tracking group, i.e., a functional group suitable for tracking delivery material in vitro and in vivo. This tracking group may contain stable isotopes of carbon (C) or hydrogen (H), such as ¹³C or ²H (also referred to herein as deuterium, D or d). When a carrier is formulated into nanoparticles with nucleic acids, such as replicon RNA, the nanoparticles may be tracked in vitro and in vivo post-administration by techniques such as mass spectroscopy or nuclear magnetic resonance imaging. Inclusion of stable isotopes may be beneficial for identification of delivery molecules since these isotopes differ from the relatively abundant ¹²C and ¹H isotopes that predominate in tissues. Tracking may be useful for identifying biodistribution, material clearance and molecular stability of nanoparticles post-administration, and related issues. Isotopically labeled fatty acids may be, but are not limited to, octanoic acid-1-¹³C, octanoic acid-8 ¹³C, octanoic acid-8,8,8-²H3, octanoic ²H15 acid, decanoic acid-1-¹³C, decanoic acid-10-¹³C, decanoic-10,10,10-²H3 acid, decanoic ²H19 acid, undecanoic acid-1-¹³C, lauric acid-12,12,12-²H3, lauric ²H23 acid, lauric acid-1-¹³C, lauric acid-1,12-¹³C2, tridecanoic-2,2-²H2 acid, myristic acid-14 ¹³C, myristic acid-1-¹³C, myristic acid-14,14,14-²H3, myristic-d27 acid, palmitic acid-1-¹³C, palmitic acid-16-¹³C, palmitic acid-16-¹³C,16,16,16-²H3, palmitic acid ²H31, stearic acid-1-¹³C, stearic acid-18-¹³C, stearic acid-18,18,18-²H3, stearic ²H35 acid, oleic acid-1-¹³C, oleic acid ²H34, linolenic acid-1-¹³C, linoleic acid ²H32, arachidonic-5,6,8,9,11,12,14,15 ²H8 acid, or eicosanoic ²H39 acid.

Referring to FIG. 1, a carrier may include a poly(ethylene glycol) (“PEG”) moiety 128. PEG, as used in this disclosure, is a linear, water-soluble polymer of ethylene repeating units with two terminal hydroxyl groups. In some cases, where specifically indicated, PEG polymers may be branched. A PEG moiety may have an average molecular weight ranging from about 100 Daltons to about 750 Daltons. In some embodiments, a PEG moiety may be from 1 to 15 monomers long. Non-limiting examples of PEGylated carriers are shown in FIGS. 2A-2L. A PEGylated carrier may have approximately 1-10 PEG chains.

Still referring to FIG. 1, a carrier may comprise a PEG alternative in place of a PEG moiety. As used herein, “PEG alternatives” refers to natural, synthetic or zwitterionic polymers as alternative candidates to PEG such as Polyglycerol (PG), polyaminoacids, polyacrylamides, Polyvinylpyrrolidone (PVP), zwitterionic polymers, polysaccharides (ex. polysialic acid, heparin, hyaluronic acid etc), Poly(N-(2-hydroxypropyl)methacrylamide) or PHPMA,poly(N,N-dimethyl acrylamide) (PDMA), poly(N-acryloylmorpholine) (PAcM), poly(oligo(ethylene glycol) methyl ether methacrylate) (POEGMA), Polysarcosine, Polyoxazoline and Polyphosphoester with degree of polymerization ranging from 1-100. In non-limiting examples, an acrylamide may include poly(N,N-dimethyl acrylamide) (PDMA) or poly(N-acryloylmorpholine) (PAcM). In non-limiting examples, a polysaccharide may include polysialic acid, heparin, or hyaluronic acid. Non-limiting examples of dendritic polymer conjugates with PEG alternatives are shown in FIGS. 2M-2O.

Still referring to FIG. 1, in an embodiment, a PEG moiety may include an additional chemical moiety 116 that may influence the physicochemical behavior of carrier as well as nanoparticle composition 100. Such groups may include, but not limited to alkyne, azide, amino (NH2), NHMe, NHBoc, Br, I, aminooxy, aryl, carboxylic acid (CO₂H), sulfonic Acid (SO₃H), NHS ester, methyl, hydroxy, phosphonate, silane, tosylate, NHPEG, NMePEG, NBocPEG and the like.

Still referring to FIG. 1, in an embodiment, a PEG moiety may have a molecular weight of less than about 50 Daltons. In an embodiment, a PEG moiety may have a molecular weight of less than about 150 Daltons. In an embodiment, a PEG moiety may have a molecular weight of less than about 250 Daltons. In an embodiment, a PEG moiety may have a molecular weight of less than about 350 Daltons. In an embodiment, a PEG moiety may have a molecular weight of less than about 450 Daltons. In an embodiment, a PEG moiety may have a molecular weight of less than about 550 Daltons. In an embodiment, a PEG moiety may have a molecular weight of less than about 650 Daltons. In an embodiment, a PEG moiety may have a molecular weight of less than about 750 Daltons. In an embodiment, a PEG moiety may have a molecular weight of less than about 850 Daltons. In an embodiment, a PEG moiety may have a molecular weight of less than about 950 Daltons. In an embodiment, a PEG moiety may have a molecular weight of less than about 1050 Daltons. In an embodiment, a PEG moiety may have a molecular weight of less than about 1150 Daltons. In an embodiment, a PEG moiety may have a molecular weight of less than about 1250 Daltons. In an embodiment, a PEG moiety may have a molecular weight of less than about 1350 Daltons. In an embodiment, a PEG moiety may have a molecular weight of less than about 1450 Daltons. In an embodiment, a PEG moiety may have a molecular weight of less than about 1550 Daltons.

Still referring to FIG. 1 n an embodiment, a PEG moiety may have a length of about 1 to 5 monomers. In an embodiment, a PEG moiety may have a length of about 1 to 6 monomers. In an embodiment, a PEG moiety may have a length of about 1 to 7 monomers. In an embodiment, a PEG moiety may have a length of about 1 to 8 monomers. In an embodiment, a PEG moiety may have a length of about 1 to 9 monomers. In an embodiment, a PEG moiety may have a length of about 1 to 10 monomers. In an embodiment, a PEG moiety may have a length of about 1 to 11 monomers. In an embodiment, a PEG moiety may have a length of about 1 to 12 monomers. In an embodiment, a PEG moiety may have a length of about 1 to 13 monomers. In an embodiment, a PEG moiety may have a length of about 1 to 14 monomers. In an embodiment, a PEG moiety may have a length of about 1 to 15 monomers. In an embodiment, a PEG moiety may have a length of about 1 to 16 monomers. In an embodiment, a PEG moiety may have a length of about 1 to 17 monomers. In an embodiment, a PEG moiety may have a length of about 1 to 18 monomers. In an embodiment, a PEG moiety may have a length of about 1 to 19 monomers. In an embodiment, a PEG moiety may have a length of about 1 to 20 monomers. In an embodiment, a PEG moiety may have a length of about 1 to 21 monomers. In an embodiment, a PEG moiety may have a length of about 1 to 22 monomers. In an embodiment, a PEG moiety may have a length of about 1 to 23 monomers. In an embodiment, a PEG moiety may have a length of about 1 to 24 monomers. In an embodiment, a PEG moiety may have a length of about 1 to 25 monomers.

Still referring to FIG. 1: In an embodiment, a PEG moiety may have a length of about 5 monomers. In an embodiment, a PEG moiety may have a length of about 6 monomers. In an embodiment, a PEG moiety may have a length of about 7 monomers. In an embodiment, a PEG moiety may have a length of about 8 monomers. In an embodiment, a PEG moiety may have a length of about 9 monomers. In an embodiment, a PEG moiety may have a length of about 10 monomers. In an embodiment, a PEG moiety may have a length of about 11 monomers. In an embodiment, a PEG moiety may have a length of about 12 monomers. In an embodiment, a PEG moiety may have a length of about 13 monomers. In an embodiment, a PEG moiety may have a length of about 14 monomers. In an embodiment, a PEG moiety may have a length of about 15 monomers. In an embodiment, a PEG moiety may have a length of about 16 monomers. In an embodiment, a PEG moiety may have a length of about 17 monomers. In an embodiment, a PEG moiety may have a length of about 18 monomers. In an embodiment, a PEG moiety may have a length of about 19 monomers. In an embodiment, a PEG moiety may have a length of about 20 monomers. In an embodiment, a PEG moiety may have a length of about 21 monomers. In an embodiment, a PEG moiety may have a length of about 22 monomers. In an embodiment, a PEG moiety may have a length of about 23 monomers. In an embodiment, a PEG moiety may have a length of about 24 monomers. In an embodiment, a PEG moiety may have a length of about 25 monomers.

Referring to FIG. 1, nanoparticle composition 100 may include other components 120 such as a lipid conjugate. A lipid conjugate may be useful in that it may prevent the aggregation of particles. Lipid conjugates that may be in a composition herein include, but are not limited to, polymer-lipid conjugates where the polymer component may be polyethylene glycol (PEG), poly(propylene glycol) (PPG), and copolymers of ethylene glycol and propylene glycol, poly(saccharides), or copolymers, and mixtures thereof. In some embodiments, the polymer component of a polymer-lipid conjugate is polyethylene glycol with a molecular weight between about 120 Da and about 16,000 Da. In some embodiments, the polymer component of a polymer-lipid conjugate is polyethylene glycol with a molecular weight of about 2,000 Da. In some embodiments, a polymer-lipid conjugate includes PEG coupled to lipids such as DMG-PEG 2000, PEG coupled to phospholipids such as phosphatidylethanolamine (PEG-PE), PEG conjugated to cholesterol or a derivative thereof, and mixtures thereof. In certain instances, PEG may be optionally substituted by an alkyl, alkoxy, acyl, or aryl group. The physical properties of a nanoparticle composition may vary depending on the lipid conjugate used. For instance, the size, relative quantity, and distribution of a PEG-lipid included in a nanoparticle composition may affect physical properties of the nanoparticle composition. The properties that can be controlled may be, but are not limited to, diameter of a nanoparticle, the propensity of the nanoparticles to aggregate, the number of nucleic acid molecules inside each nanoparticle, or the concentration of the nanoparticles in a nanoparticle composition, the efficacy of intra-cellular delivery of therapeutic and immunogenic nucleic acid agents, and/or the efficacy of uptake of the nanoparticles by cells.

Still referring to FIG. 1, in some embodiments, a nanoparticle composition may contain 10 mol % or less of a PEG-lipid conjugate. A nanoparticle composition may include about 10 mol %, about 9 mol %, about 8 mol %, about 7 mol %, about 6 mol %, about 5 mol %, about 4 mol %, about 3 mol %, about 2 mol %, or about 1 mol %, or any amount in between any two of the foregoing integers of a PEG-lipid conjugate. A nanoparticle composition containing a PEG-lipid conjugate may include nanoparticles with a smaller diameter than nanoparticles of the composition lacking the PEG-lipid conjugate.

Referring to FIG. 1, in an embodiment, nanoparticle composition 100 may include other components 120 such as an amphipathic liquid. As used in this disclosure, an “amphipathic lipid” is any lipid having non-polar hydrophobic “tails” and polar “heads.” Polar groups may include, but are not limited to, phosphate, carboxylic, sulfato, amino, sulfhydryl, nitro, hydroxyl, or other like groups. Nonpolar groups may include, but are not limited to, long-chain saturated, and unsaturated aliphatic hydrocarbon groups and such groups substituted by one or more cycloalkyl, cycloalkenyl, aryl, heteroaryl, or heterocycle group(s). Examples of amphipathic lipids include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidyl choline, dioleoylphos-phatidylcholine, di stearoylphosphatidylcholine, and dilinoleoylphosphatidylcholine. In some embodiments, an amphipathic lipid is 1,2-dioleoyl-snglycero-3-phosphoethanolamine (DOPE). In some embodiments, an amphipathic lipid is distearoylphosphatidylcholine (DSPC). In some embodiments, a nanoparticle composition may contain an amphipathic lipid in an amount ranging from 10 mol % to 25 mol %.

Referring to FIG. 1, in an embodiment, nanoparticle composition 100 may include other components 120 such as cholesterol or a cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. A cholesterol derivative may include a sugar moiety and/or amino acids such as serine, threonine, lysine, histidine, arginine or their derivatives. A nanoparticle composition may include a cholesterol or cholesterol derivative in an amount ranging from 50 mol % to 75 mol %.

Referring to FIG. 1, in an embodiment, a nanoparticle composition comprising at least one of the carriers described herein is provided. A nanoparticle composition herein may be used to introduce an agent, such as a nucleic acid agent, into a cell. A nanoparticle composition herein may be useful as a vaccine, therapeutic, or transfection agent. A nanoparticle composition herein may be useful in a method of treating or preventing a disease.

Still referring to FIG. 1, in an embodiment, a nanoparticle composition may include a mixture of carriers. In an embodiment, such a mixture comprises carriers with different amine linkers. In an embodiment, such a mixture comprises carriers with different hydrophobic groups. In an embodiment, such a mixture comprises carriers with different PEG moieties. In an embodiment, such a mixture comprises carriers with different dendritic polymer skeletons. Carriers may be mixed at a fixed ratio. As an example of a mixture with three carriers, a ratio of the first carrier to the second carrier and to the third carrier may be i:j:k where i, j and k are independently selected from 1, 2, 3, 4, 5, 6, 7, 8, 9, 10, 11, 12, 13, 14, 15, 16, 17, 18, 19, or 20, or a value between any two of the foregoing.

Still referring to FIG. 1, in an embodiment, a nanoparticle composition may include a pharmaceutically acceptable carrier. As used herein, “excipient” and “pharmaceutically-acceptable carrier” are interchangeably used to refer to a pharmaceutically acceptable material, composition or vehicle involved in carrying or transporting a payload from one cell-type, organ, or portion of the body, to another cell-type, organ, or portion of the body. Pharmaceutically-acceptable carriers include, for example, solvents, dispersion media, coatings, antibacterial and antifungal agents, isotonic and absorption delaying agents, liquid or solid fillers, diluents, excipients, manufacturing aids (such as lubricants, talc magnesium, calcium or zinc stearate, or steric acid), or solvent encapsulating materials. Each pharmaceutically-acceptable carrier may be “acceptable” in the sense of being compatible with the other ingredients of the formulation and not injurious to the subject. Some examples of materials which may serve as pharmaceutically-acceptable carriers include, without limitation: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (I8) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants.

Now referring to FIGS. 2K and 2L, an exemplary formula of pegylated dendrimer with PEG moiety and fatty acid side chains on one side and amine on the other side of the dendrimer are shown. In this figure, the fatty acid side chain may be selected from any one of C4-C28 fatty acids. The amine may be selected from the following moieties: 1-(2-Aminoethyl)piperazine, 1-Piperidineethanamine, 1-Dimethylamino-2-propylamine, 1-(3-Aminopropyl)pyrrolidine, 1-(2-Aminoethyl)pyrrolidine, 1-(2-Aminoethyl)piperidine, 2-(1-Piperazinyl)ethylamine, 2-(4-Methyl-piperazin-1-yl)-ethylamine, 3-(Dimethylamino)-1-propylamine, 3-(Diethylamino)propylamine, (4-Aminobutyl)dimethylamine, 4-(1-Pyrrolidinyl)-1-butylamine, 4-Morpholineethanamine, 4-Morpholinepropanamine, 4-Methyl-1-piperazineethanamine, 4-(Diethylamino)butylamine, N,N-DimethylethylenediamineN,N-Dimethyldipropylenetriamine, N,N′-Dimethyl-N,N′-bis(3-methylaminopropyl)trimethylenediamine, N1-(2-aminoethyl)-N1-methylpropane-1,3-diamine, N1-(3-aminopropyl)-N1-methylbutane-1,4-diamine, N1-(4-aminobutyl)-N1-methylbutane-1,4-diamine, N1-(3-aminopropyl)propane-1,3-diamine, N1-(3-aminopropyl)-N1-methylpropane-1,3-diamine, 3-((3-aminopropyl)(methyl)amino)propan-1-ol, 1-Dimethylamino-2-propanol, 1-[Bis[3-(dimethylamino)propyl]amino]-2-propanol, 2-Dimethylaminoethanol, 2-(Diethylamino)ethanol, 2-[2-(Dimethylamino)ethoxy]ethanol, 2-(2-Aminoethoxy)ethanol, 2-{[2-(Dimethylamino)ethyl]methylamino}ethanol, 3-Dimethylamino-1-propanol, 3-Diethylamino-1-propanol, 4-(Dimethylamino)-1-butanol, 4-Diethylamino-2-butyn-1-ol, N-Methyldiethanolamine, 4-(diethylamino)butan-1-ol, 3-(azetidin-1-yl)propan-1-ol, 3-(pyrrolidin-1-yl)propan-1-ol, 3-(piperidin-1-yl)propan-1-ol, 4-(azetidin-1-yl)butan-1-ol, 4-(pyrrolidin-1-yl)butan-1-ol, 4-(piperidin-1-yl)butan-1-ol, 3-morpholinopropan-1-ol, 4-morpholinobutan-1-ol, 3-(4-methylpiperazin-1-yl)propan-1-ol, 4-(4-methylpiperazin-1-yl)butan-1-ol.

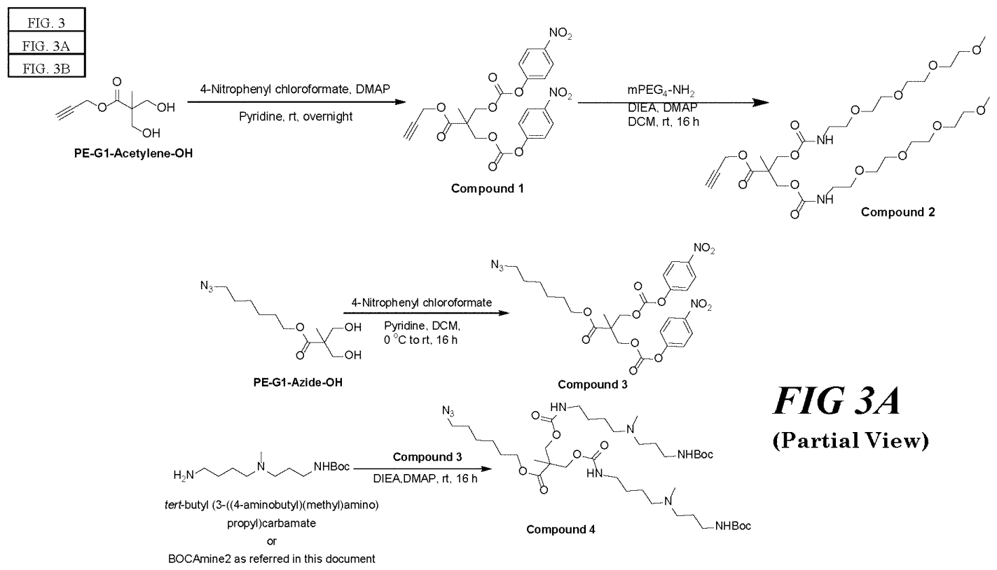

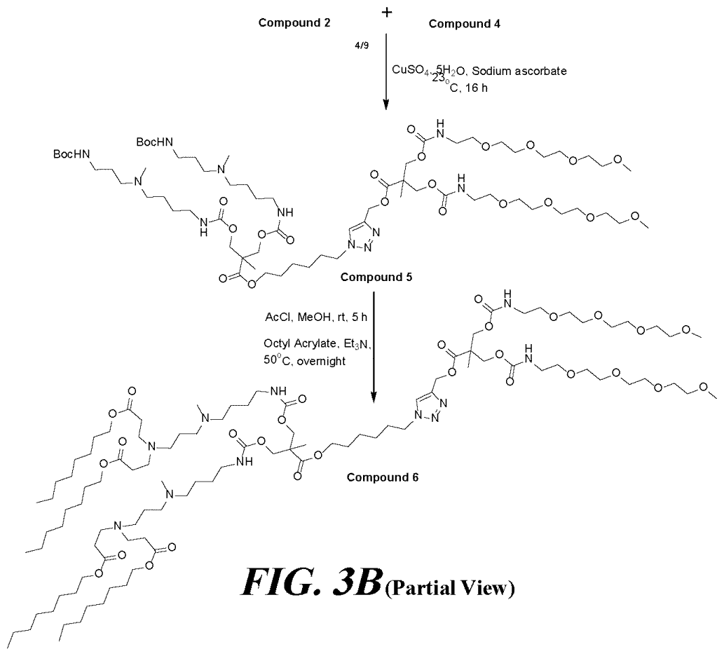

Now referring to FIG. 3, an exemplary synthetic scheme of an asymmetrical PEGylated dendrimer is shown. FIG. 3 is a schematic drawing of the synthesis of pegylated dendrimer with fatty acid side chains that may be used for helping with self-assembly.

Compound 1: PE-G1-acetylene-OH (641 mg, 3.73 mmol, MW 172.18) was dissolved in dry methylene chloride (“DCM”) (6 mL) and pyridine (5.4 ml, 30 mmol) was added followed by p-nitrophenyl chloroformate (2.0 g, 10 mmol, MW 201.6, 2.7 eq) dissolved in dry methylene chloride (15 mL). The reaction was stirred at 0° C. to room temperature for 16 h. Next day, the reaction mixture was diluted with 1.33 M NaHSO₄ and extracted with DCM. The organic layer was washed with brine, concentrated using a rotary evaporator, and purified by flash chromatography with methylene chloride/ethyl acetate. The compound eluted at methylene chloride, (R_{f}=0.7 in 20:1 methylene chloride/ethyl acetate). The yield of the reaction 840 mg (45%).

Compound 2: A solution of Compound 1 (200 mg, 0.40 mmol, MW502.39) dissolved in dry DCM (3 mL) was added to an excess of amine, mPEG4-Amine (248 mg, 1.195 mmol) dissolved in dry DCM (3 mL). A solution of 4-Dimethylaminopyridine (“DMAP,” 97 mg, 0.8 mmol) and N,N-Diisopropylethylamine (“DIPEA,” 0.28 ml, 1.6 mmol) in dry DCM (1 mL) was added and the reaction mixture was stirred 16 h at 23° C. under an argon atmosphere. Next day, the solvent was then removed in vacuo, purified via flash chromatography on silica column (12 g) with gradient elution from 100% methylene chloride (mobile phase a) to 75:22:3 methylene chloride/methanol/ammonium hydroxide (by volume, mobile phase b) over 40 minutes. The desired product eluted at 22% mobile phase b. (R_{f}=0.7 in 1:2 mobile phase a/mobile phase b) to yield the desired product as clear oil (132 mg, 52%). MS (ESI) calculated for C28H50N2O14 [M]+m/z 638.33, found 638.50.

Compound 3: PE-G1-azide-OH (250 mg, 0.96 mmol, MW 259.3) was dissolved in dry DCM (3 mL) and pyridine (0.6 ml, 7.68 mmol, 8 eq) was added followed by p-nitrophenyl chloroformate (525 mg, 2.61 mmol, MW 201.6, 2.7 eq) dissolved in dry DCM (8 mL). Next day, the reaction mixture was diluted with 1.33 M sodium bisulfate and extracted with DCM. The organic layer was washed with brine, concentrated using a rotary evaporator and purified by flash chromatography with methylene chloride/ethyl acetate. The compound eluted at 2% ethyl acetate/methylene chloride, (R_{f}=0.8 in 5:1 DCM/EtOAc). The yield of the reaction 190 mg (33%).

Compound 4: A solution of Compound 3 (200 mg, 0.34 mmol, MW 589.51) dissolved in dry DCM (1 mL) was added to an excess of amine, tert-butyl (3-((4-aminobutyl)(methyl)amino)propyl)carbamate or “BocAmine2” (200 mg, 0.77 mmol) dissolved in dry DCM (1 mL). A solution of DMAP (62 mg, 0.51 mmol) and DIPEA (0.18 mmol, 1.16 mmol) in dry DCM (1 mL) was added and the reaction mixture was stirred 16 h at 23° C. under an argon atmosphere. Next day, the solvent was then removed in vacuo, purified via flash chromatography on silica column (12 g) with gradient elution from 100% CH2C12 (mobile phase a) to 75:22:3 methylene chloride/methanol/ammonium hydroxide (by volume, mobile phase b) over 40 minutes. The desired product eluted at 24% mobile phase b (R_{f}=0.2 in 1:1 mobile phase a/mobile phase b to yield the desired product as yellow oil (173 mg, 62%).

Compound 5: To a 50 ml round bottom flask, CuSO₄·5H2O (4.5 mg, 0.013 mmol, 20 mol %, MW 249.69) and sodium ascorbate (7.2 mg, 0.026 mmol, 40 mol %, MW 198.11) were taken. Azide, compound 4 (MW: 830.23, 75 mg, 0.09 mmol) dissolved in 0.6 ml tetrahydrofuran (“THF”), then Alkyne, PE-G2-acetylene-PEG4 (MW: 638.71, 58 mg, 0.09 mmol) dissolved in THF (0.6 mL) was added along with and degassed THF: H2O (2 mL, 1:1). The reaction mixture was stirred at 23° C. for 16 h. Next day, thin layer chromatography was used to confirm completion of the reaction. The reaction mixture was purified via flash chromatography on 24 g silica column with gradient elution from 100% methylene chloride (mobile phase a) to 75:24:6 methylene chloride/methanol/ammonium hydroxide (by volume, mobile phase b). The desired product eluted at 50% mobile phase b. (R_{f}=0.6 in 1:1 mobile phase a/mobile phase b) to yield the compound 5 as yellow oil (66 mg, 50%). MS (ESI) calculated for C82H149N15O32 [M+2H]²⁺ m/z 929.1, found 929.2.

Compound 6: 54 mg of compound 5 (0.037 mmol, MW 1468) was treated with 20 eq of AcC1 (0.05 ml, 0.74 mmol, 20 eq) after dissolving the compound in 3 ml MeOH, the reaction was stirred at 0° C. to 23° C. for 5 h, evaporated to dryness and dissolved in 0.5 ml DMF, added 0.1 ml Et₃N (1.27 mmol, 20 eq) followed by 0.05 ml of octyl acrylate (0.22 mmol, MW 184.3 g/mol, d 0.88 g/ml). The reaction mixture was stirred at 50° C. for 24 h, and the reaction mixture was purified via flash chromatography on silica column (24 g) with gradient elution from 100% CH2C12 (mobile phase a) to 75:22:3 CH2C12/MeOH/NH₄OHaq (by volume, mobile phase b). The desired product eluted at 40% mobile phase b. (R_{f}=0.5 in 1:1 mobile phase a/mobile phase b) to yield the desired product as yellow oil (10 mg, 13% over two steps) as clear oil. MS (ESI) calculated for C101H191N11O28 [M+2H]²⁺ m/z 1003.19, found 1003.5.

Now referring back to FIG. 1, nanoparticles containing the PEGylated dendrimer (e.g PE G1 PEG4-PE G1 A2-4 Octyl):DOPE:cholesterol:DMG-PEG2k at molar ratios of 1:0.75:3.6:0.1 were formulated using NanoAssemblr Benchtop (Precision NanoSystems Inc, Vancouver, BC, Canada). RNA was diluted with DNase/RNase-Free, endotoxin free distilled water and sterile citrate buffer pH 6.0 to a final desired pH. Total flow rate was maintained at 10 mL per min at a 3:1 ratio of aqueous to organic phase for formulating on the Benchtop. Using glassware depyrogenated by heating at 250° C. for 24 hour, nanoparticles were dialyzed against sterile, endotoxin-free PBS using 20,000 molecular weight cutoff dialysis. Dialyzed nanoparticles were sterile filtered using 0.2 micron poly(ether sulfone) filters and characterized with a Zetasizer NanoZS machine (Malvern). The size distributions were characterized by a single peak with a low polydispersity index. Encapsulation efficiency was measured to be 95% for the nanoparticle composition containing PE G1 PEG4-PE G1 A2-4 Octyl and SEAP replicon RNA using Ribogreen® assay (Geall et al. 10.1073/pnas.1209367109 which is incorporated herein by reference as if fully set forth).

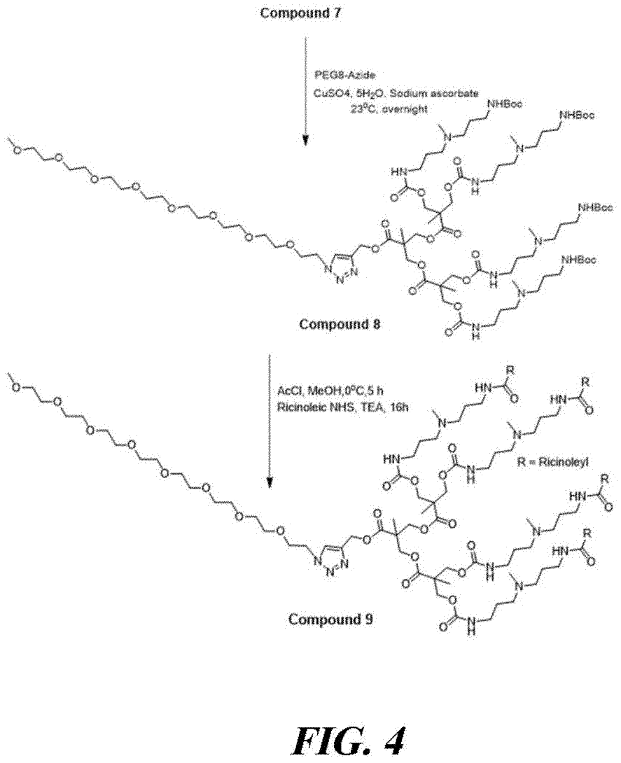

Now referring to FIG. 4, an exemplary synthetic route to a nanoparticle composition containing a pegylated dendron is shown. Dendritic-linear polymer conjugation was demonstrated by a convergent synthesis approach starting from an alkyne-terminated dendron that was subsequently coupled with azide-terminated poly(ethylene glycol) by a robust, and efficient methodology.

Compound 8: PEG8 Azide (MW: 409.5, 100 mg, 0.24 mmol, 1.5 eq) was taken in 50 ml round bottom flask, dissolved in 1 ml THF, then 200 mg of Compound 7, (PE-G2-acetylene-BocAmine1, MW:1489, 0.13 mmol, synthesized following published procedure: Talukder et al., International Application Number WO 2021207020, entitled, “Carriers for Efficient Nucleic Acid Delivery,” filed on Feb. 4, 2021, which is incorporated herein by reference in its entirety) dissolved in THF (2 mL) was added along with CuSO₄·5H2O (3.3 mg, 0.013 mmol, 10 mol %, MW 249.69) and sodium ascorbate (5.1 mg, 0.026 mmol, 20 mol %, MW 198.11), and degassed THF: H2O (2 mL, 1:1). The reaction mixture was stirred at 23° C. for 16 h. Next day, the reaction was deemed completed by using thin layer chromatography. The reaction mixture was purified via flash chromatography on 12 g silica column with gradient elution from 100% CH₂Cl₂ (mobile phase a) to 75:22:3 CH2C12/MeOH/NH₄OHaq (by volume, mobile phase b). The desired product eluted at 100% mobile phase b. (R_{f}=0.7 in 2:1 mobile phase b/mobile phase a) to yield the compound 8 as yellow oil (140 mg, 60%). MS (ESI) calculated for C₈₇H165N15O30 [M+2H]²⁺ m/z 950.1, found 949.1.

Compound 9: 80 mg of compound 8 (0.042 mmol) was treated with 20 eq of acetyl chloride (“AcC1,” 0.06 ml, 0.84 mmol) after dissolving the compound in 3 ml MeOH, the reaction was stirred at 0° C. to 23° C. for 5 h, evaporated to dryness and dissolved in 2 ml DMF, added 0.12 ml Et₃N (0.84 mmol, 20 eq) followed by 100 mg of Ricinoleic-NHS (as synthesized following published procedure: Talukder et al., International Application Number WO 2021207020, entitled, “Carriers for Efficient Nucleic Acid Delivery,” filed on Feb. 4, 2021, which is incorporated herein by reference in its entirety) dissolved in 2 ml DMF. The reaction mixture was stirred at 23° C. for 24 h, concentrated under reduced pressure in Genevac, and the reaction mixture was purified via flash chromatography on silica column (12 g) with gradient elution from methylene chloride (mobile phase a) to 75:24:6 methylene chloride/methanol/ammonium hydroxide (by volume, mobile phase b). The desired product eluted at 40% mobile phase b. (R_{f}=0.4 in 2:1 mobile phase b/mobile phase a) to yield the desired product as yellow oil (12 mg, 11% over two steps). MS (ESI) calculated for C139H261N15O30 [M+2H]²⁺ m/z 1310.0, found 1308.9.

Additionally, with reference to FIG. 4, nanoparticles were formulated by mixing 30 μl of an ethanol phase containing PEG-modified dendron (Compound 9) and DMG PEG 2000 (PEG-lipid, Avanti Polar Lipids) with 90 μl of Replicon RNA encoding secreted embryonic alkaline phosphatase (SEAP) diluted with ultraPure, DNase/RNase-free, endotoxin-free distilled water (Invitrogen) and sterile 100 mM (pH 4.5) acetate buffer to a final citrate concentration of 10 mM. The resulting nanoparticles contained 13:1:4 mass ratio of carrier to PEG-lipid to RNA. Formulations were diluted 500-fold for analysis of particle size distribution, Z-average, and derived count rate using a Zetasizer Nano ZS (Malvern Panalytical).

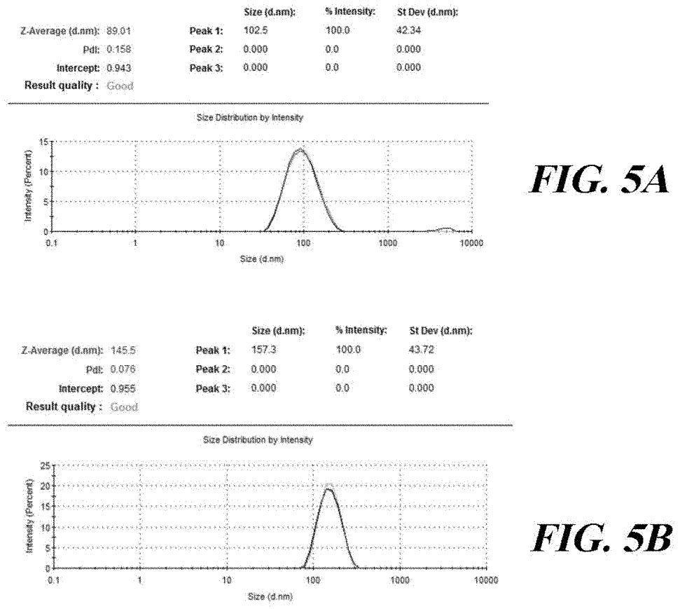

Now referring to FIG. 5, a distribution of the nanoparticle composition measured as the random change in intensity of light (Z average) based on size (d.nm; diameter in nm) of the nanoparticles is shown. The “Z average” of the nanoparticle composition containing PE G1 PEG4-PE G1 A2-4 Octyl or PE Dendron_G2-PEG8-A1-Ricinoleic and SEAP Replicon RNA as function of size was determined by dynamic light scattering (DLS). Referring to FIGS. 5A and 5B, the strongest intensity was observed for the nanoparticles of 102.5 d.nm in size and 157.3 d.nm in size respectively. The size distributions were characterized by a single peak with a low polydispersity index, indicating a relatively monodisperse size.

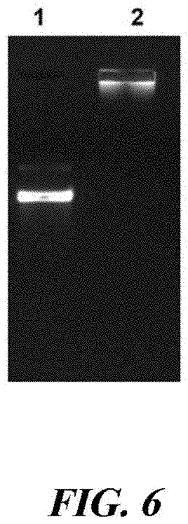

Now referring to FIG. 6, exemplary measurements obtained from gel well-retention assay are shown with a photograph of the agarose gel showing the binding of the PEGylated dendron with RNA. Known methods, such as Geall et al. 10.1073/pnas.1209367109 were used. The gels were stained with ethidium bromide (EB) and gel images were taken on a Syngene G Box imaging system (Syngene, USA). Lane 1 contained the unformulated SEAP replicon RNA, lane 2 contained the product of formulation of the Compound 9 and SEAP replicon RNA. Before loading, the samples were incubated with formaldehyde loading dye, denatured for 10 min at 65° C. and cooled to room temperature. The gel was run at 90 V and gel images were taken on a Syngene G Box imaging system (Syngene, USA). The lower band corresponds to the small size free RNA (lane 1) and the top band (lane 2) represent the large size nanoparticles formed by binding of the RNA to the PEG-modified dendron carrier.

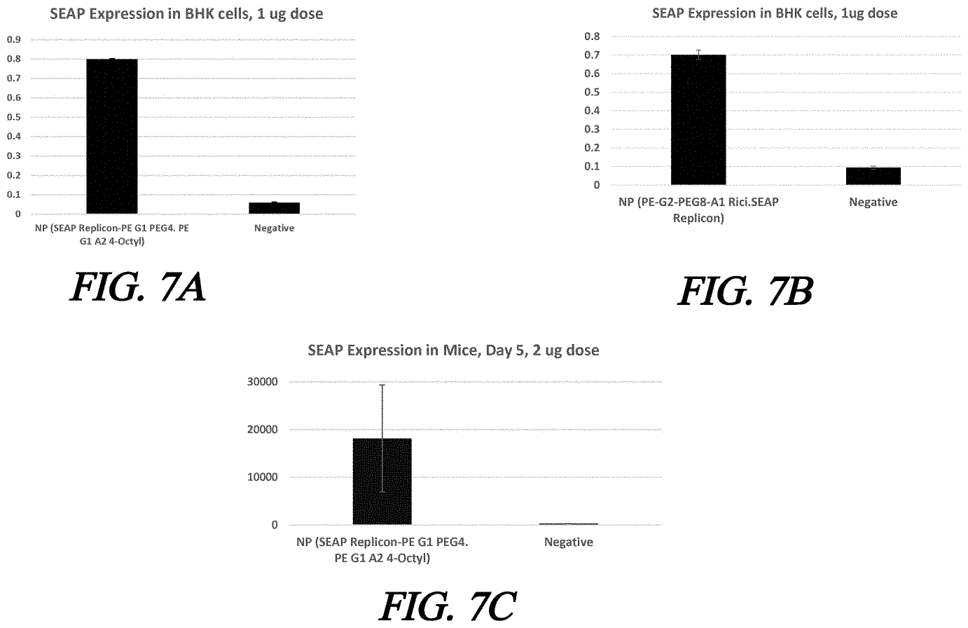

Now referring to FIG. 7, optical density measurements showing the expression of secreted alkaline phosphatase (SEAP) with nanoparticle formulations using PEGylated dendrimer, PE G1 PEG4-PE G1 A2-4 Octyl and PEGylated dendron, PE Dendron_G2-PEG8-A1-Ricinoleic is shown. To test the ability of the nanoparticles formulated with PEGylated carrier to express SEAP in vitro, BHK cells were treated with nanoparticles. Each well of a 12 well dish of BHKs was treated with 20 μL (approximately 1 μg) of each formulation product diluted into a final volume of 500 μL with a 1:1 Optimem:PBS mix. After the treatment, BHK cells were incubated at 37° C. and 5% CO2. After 12, hours, cell culture medium was collected and assayed for SEAP using the InvivoGen QUANTI-Blue™ detection system (San Diego, CA, USA), according to the manufacturer's protocol. Briefly, 50 μL of the cell culture medium was added to 150 μL of the QUANTI-Blue™ solution and incubated at 37° C. for 10 minutes. The Optical Density (OD) was measured at 620-655 nm using a microplate reader. FIGS. 7A and 7B illustrate the SEAP expression of nanoparticle formulations using PE G1 PEG4-PE G1 A2-4 Octyl and PE Dendron_G2-PEG8-A1-Ricinoleic based on optical density compared to the negative control.

Now referring to FIG. 7C, to test the ability of the nanoparticles formulated with PEGylated carrier to express SEAP in vivo, mice were injected with nanoparticles at a dose of 2 ug of SEAP replicon RNA, and 16 hrs later, serum was collected from the mice. The amount of SEAP was quantified using the Invitrogen NovaBright™ Phospha-Light™ EXP Assay kits for SEAP detection according to the manufacturer's protocol. The amount of SEAP in the mouse serum samples are reported in Arbitrary Units (A.U.) as measured in a BioTek Synergy HTX microplate reader. Error bars are ±S.E.M. Referring to FIG. 7C, illustrates the SEAP expression of nanoparticle formulation using PE G1 PEG4-PE G1 A2-4 Octyl.

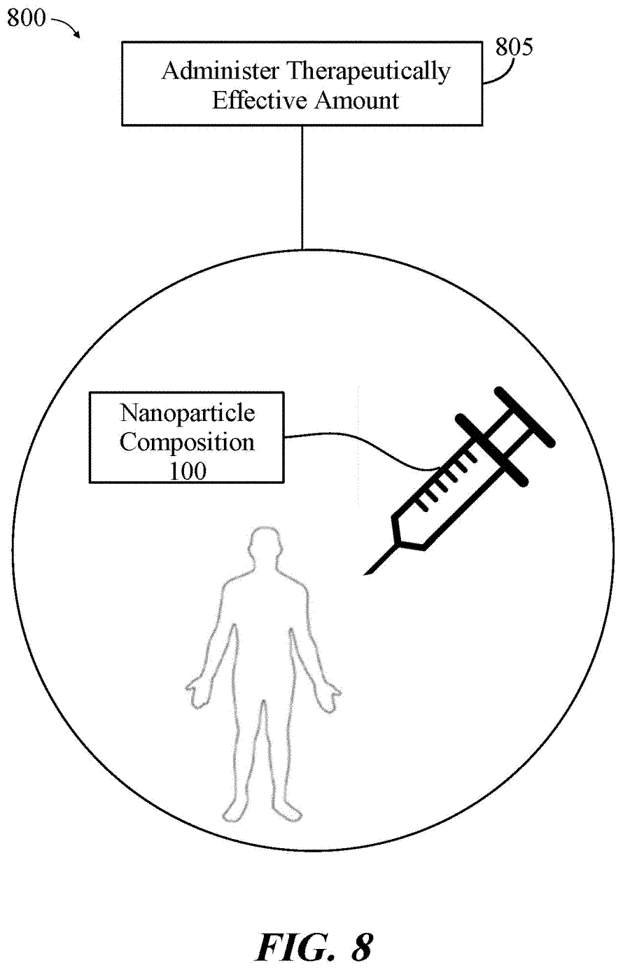

Referring to FIG. 8, an exemplary method 800 for treating or preventing a disease or condition in a subject is disclosed. At step 805, a therapeutically effective amount of nanoparticle composition 100 is administered to a subject. As an example of this method, a therapeutically effective amount of a nanoparticle composition may be administered to induce immune tolerance, for instance, to reduce a subject's immune responses against an encoded antigen, to treat allergic or autoimmune diseases, or the like.

Still referring to FIG. 8, a therapeutically effective amount of a nanoparticle composition may be administered to a subject. As used herein, a “therapeutically effective amount” is an amount sufficient to reduce or eliminate any symptom of a disease in a subject. For example, when treating viral infection, a decrease of disease symptoms may be assessed by a decrease of virus in feces, in bodily fluids, or in secreted products. The exact dosage may be chosen by a clinician based on a variety of factors and in view of individual patients. Dosage and administration may be adjusted to provide sufficient levels of an active agent or agents or to maintain a desired effect. Factors which may be taken into account may include without limitation the type and severity of a disease; age and gender of the patient; drug combinations; and an individual response to therapy. Therapeutic efficacy and toxicity of active pharmaceutical agents in a nanoparticle composition may be determined by standard pharmaceutical procedures, for example, by determining the therapeutically effective dose in 50% of the population (ED50) and the lethal dose to 50% of the population (LD50) in cells cultured in vitro or experimental animals. Nanoparticle compositions may be evaluated based on the dose ratio of toxic to therapeutic effects (LD50/ED50), called the therapeutic index, the large value of which may be used for assessment. Data obtained from cell and animal studies may be used in formulating a dosage for human use. The therapeutically effective dose may be estimated initially from cell culture assays. A therapeutically effective dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay. The amount of particles administered will depend upon which therapeutic agent (e.g., nucleic acid) is used, which disease or disorder is being treated, the age, weight, and condition of the patient, and the judgment of the clinician. A therapeutically effective dose may be between 0.001 m and 50 mg of the therapeutic or immunogenic nucleic acid per kilogram of body weight of the subject. A combination of different nucleic acid agents may be used per treatment dose.

Still referring to FIG. 8, in some embodiments, nanoparticle compositions may be administered in an amount effective to decrease the amount of expression of a target gene, or to prevent or decrease the serum concentration of a target gene product in a subject. In some embodiments, nanoparticle compositions may be administered in an amount effective to increase the amount of expression of a gene, such as a gene encoded by a nucleic acid agent, or to increase the serum concentration of a gene product in a subject. In some embodiments, nanoparticle compositions may be administered in an amount effective to stimulate a primary immune response to an antigen in a subject. In some embodiments, nanoparticle compositions may be administered in an amount effective to induce presentation of an antigen by antigen-presenting cells. In some embodiments, nanoparticle compositions may be administered in an amount that does not induce significant cytotoxicity in the cells of a subject compared to an untreated control subject.

Still referring to FIG. 8, a nanoparticle composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration. As used herein, the terms “administer,” “administering,” “administration,” or the like refer to the placement of a composition into a subject by any method. In an embodiment, a composition described herein may be administered by intravenous infusion or injection. Injection includes, without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebral, and intrasternal injection and infusion. Exemplary modes of administration include, but are not limited to, injection, infusion, instillation, inhalation, or ingestion. Administration may utilize a method or route which results in at least partial localization of a composition at a desired site, potentially increasing efficacy or reducing off target effects.

Still referring to FIG. 8, therapeutic efficacy may depend on the timing of administration of a disclosed composition. Administering a nanoparticle composition may be a preventive measure. Administering a nanoparticle composition may be a therapeutic measure, such as to promote immunity to an infectious agent, to minimize complications associated with the slow development of immunity especially in patients with a weak immune system, the elderly, or infants.

Still referring to FIG. 8, potential subjects may include without limitation, humans and animals. For example, a subject may be a vertebrate such as a primate, rodent, domestic animal or game animal. Primates may include, as a non-limiting example, chimpanzees, cynomolgus monkeys, spider monkeys, and macaques, e.g., Rhesus. A rodent may be selected from mice, rats, guinea pigs, woodchucks, ferrets, rabbits and hamsters. Domestic or game animals may be selected from cows, horses, pigs, deer, bison, buffalo, feline species, such as, but not limited to domestic cat, canine species, such as, but not limited to dog, fox, wolf, avian species, such as, but not limited to chicken, emu, ostrich, and fish, such as, but not limited to trout, catfish and salmon. In an embodiment, a subject may be a mammal, such as a primate or a human. A subject may be a mammal. A mammal may be a human, non-human primate, mouse, rat, dog, cat, swine, horse, cow, or swine but is not limited to these examples. Mammals other than humans may be subjects that represent animal models of a disease or disorder. In addition, the methods described herein may be directed to treating domesticated animals such as pets.

Still referring to FIG. 8, a nanoparticle composition may deliver nucleic acid agents to a subject in an amount effective to vaccinate the subject from one or more diseases and disorders. Nanoparticle compositions may serve as a vaccination platform for, without limitation, cancer or microbial pathogens, such as bacterial, viral, fungal and protozoan pathogens.

Still referring to FIG. 8, in certain embodiments, nanoparticle compositions may be used to immunize a subject against cancer. Nanoparticle compositions may be administered to a subject diagnosed with cancer (i.e., as a therapeutic vaccine), or to a subject having a predisposition or risk of developing cancer (i.e., as a prophylactic vaccine). In some embodiments, a nanoparticle composition may be administered to a cancer patient in addition to one or more additional therapeutic agents. In some embodiments, bioinformatics may be used to sequence each patient's unique tumor exome to identify neoantigens. Then, corresponding mRNAs of these neoantigens may be used to generate the antigens necessary to create immunity. Finally, these mRNAs may be delivered via a nanoparticle composition.

Still referring to FIG. 8, in some embodiments, a nanoparticle composition may include nucleic acid agents encoding one or more tumor antigens. A nanoparticle composition may be used to provide immunity and therapeutic activity against tumor cells and non-tumor cells located within a tumor or a tumor environment. Nanoparticle compositions may provide protective and/or therapeutic activity against solid tumors and cancers of the blood. Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma.

Still referring to FIG. 8, nanoparticle compositions may deliver nucleic acid agents encoding antigens to a subject in an amount effective to vaccinate the subject from one or more infectious diseases caused by a wide variety of microbial pathogens, such as bacterial, viral, fungal and protozoan pathogens. In some embodiments, the target of a vaccine could be any of a large number of microbial pathogens. Exemplary diseases that can be vaccinated against include disease for which vaccines are currently available, including Anthrax; Diseases (e.g., cervical cancer, cancer of the esophagus) caused by Human Papillomavirus (HPV); Diphtheria; Hepatitis A; Hepatitis B; Haemophilus influenzae type b (Hib); Influenza viruses (Flu); Japanese encephalitis (JE); Lyme disease; Measles; Meningococcal; Monkeypox; Mumps; Pertussis; Pneumococcal; Polio; Rabies; Rotavirus; Rubella; Shingles (Herpes Zoster); Smallpox; Tetanus; Toxoplasmosis; Typhoid; Tuberculosis (TB); Varicella (Chickenpox); Yellow Fever. In some embodiments, nanoparticle compositions may be used to immunize a subject against an infectious disease or pathogen for which no alternative vaccine is available, such as diseases including, but not limited to, malaria, streptococcus, Ebola Zaire, HIV, Herpes virus, hepatitis C, Middle East Respiratory Syndrome (MERS), Sleeping sickness, Severe Acute Respiratory Syndrome (SARS), rhinovirus, chicken pox, hendra, NIPA virus, Zika Virus, and others.

Referring to FIG. 8, nanoparticle compositions may be administered alone, or in combination with one or more additional active agent(s), as part of a therapeutic or prophylactic treatment regime. A nanoparticle composition may be administered on the same day, or a different day than a second active agent. As used herein, “combination” or “combined” means either concomitant, simultaneous, or sequential administration of two or more agents. In some embodiments, combinations may be administered concomitantly (e.g., as a mixture), separately but simultaneously (e.g., via separate intravenous lines into the same subject), or sequentially (e.g., one compound or agent is given first followed by a second compound or agent). In some embodiments, an additional prophylactic or therapeutic agent may be a vaccine for a specific antigen, which may be the same or different than an antigen encoded by a nucleic acid agent.

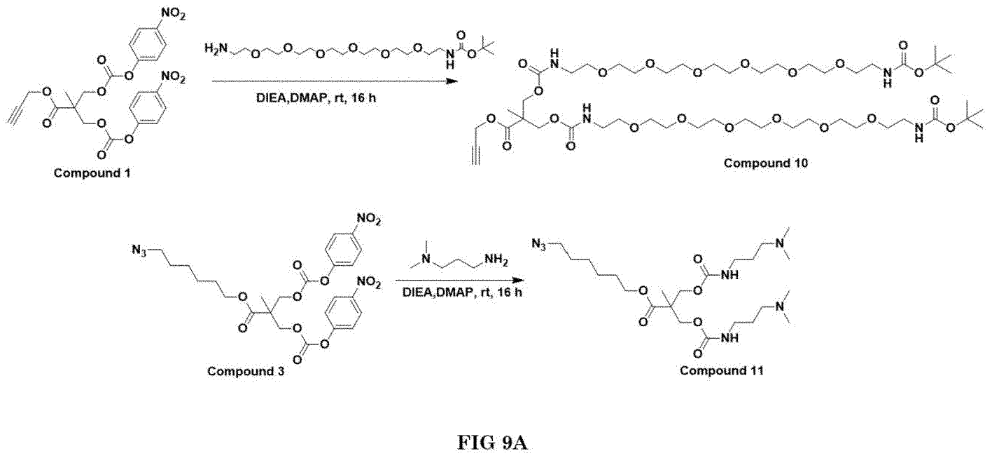

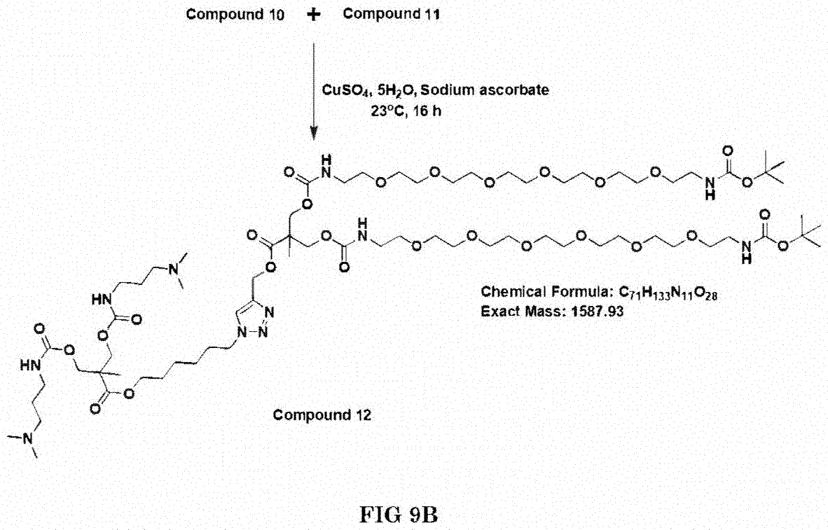

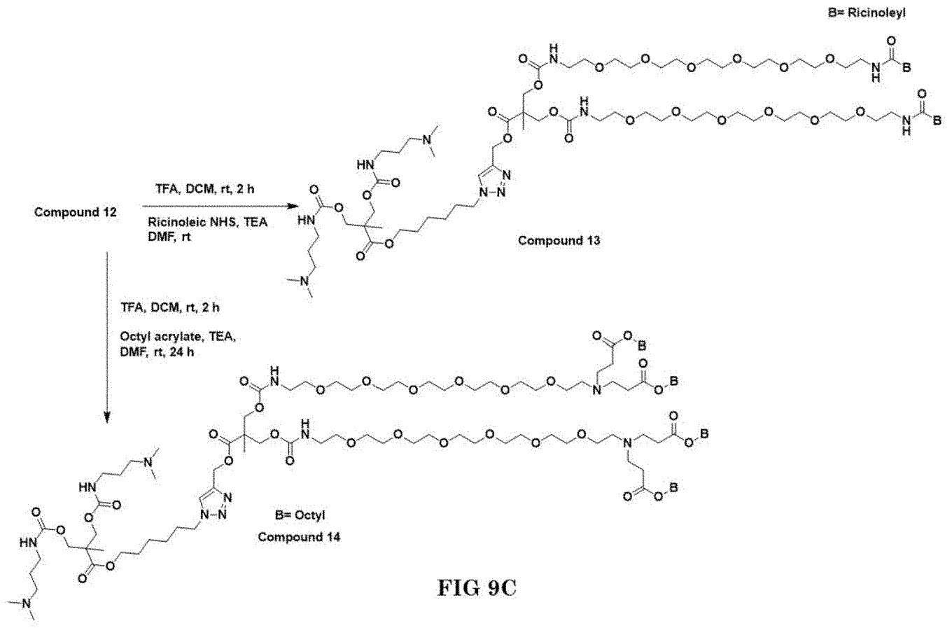

Now referring to FIG. 9, an exemplary synthetic scheme of an asymmetrical PEGylated dendrimer is shown. FIG. 9 is a schematic drawing of the synthesis of pegylated dendrimer with fatty acid side chains that can be used for helping with self-assembly. In this figure, the fatty acid side chain may be selected from any one of C₄-C₂₈ fatty acids.

Compound 10: A solution of Compound 1 (67 mg, 0.133 mmol, MW502.39) dissolved in dry DCM (3 mL) was added to an excess of amine, NH2-PEG6-NHBoc (170 mg, 0.4004 mmol) dissolved in dry DCM (3 mL). A solution of DMAP (33 mg, 0.266 mmol) and DIPEA (0.09 ml, 0.53 mmol) in dry DCM (1 mL) was added and the reaction mixture was stirred 16 h at 23° C. under an argon atmosphere. Next day, the solvent was removed in vacuo, purified via flash chromatography on silica column (24 g) with gradient elution from 100% methylene chloride (mobile phase a) to 75:22:3 methylene chloride/methanol/ammonium hydroxide (by volume, mobile phase b) over 90 minutes. The desired product eluted at 19% mobile phase b. (R_{f}=0.55 in 1:1 mobile phase a/mobile phase b) to yield the desired product as clear oil (108 mg, 76%). MS (ESI) calculated for C48H88N4O22 [M+NH4]+m/z 1090.6, found 1090.50.

Compound 11: A solution of Compound 3 (200 mg, 0.34 mmol, MW 589.51) dissolved in dry DCM (1 mL) was added to an excess of amine, 3-(dimethylamino)-propylamine (100 uL, 0.78 mmol) dissolved in dry DCM (1 mL). A solution of DMAP (62 mg, 0.51 mmol) and DIPEA (0.2 mL, 1.16 mmol) in dry DCM (1 mL) was added and the reaction mixture was stirred 16 h at 23° C. under an argon atmosphere. Next day, the solvent was then removed in vacuo, purified via flash chromatography on silica column (40 g) with gradient elution from 100% CH2C12 (mobile phase a) to 75:22:3 methylene chloride/methanol/ammonium hydroxide (by volume, mobile phase b) over 70 minutes. The desired product eluted at 70% mobile phase b (R_{f}=0.3 in mobile phase b to yield the desired product as yellow oil (144 mg, 82%). MS (ESI) calculated for C₄₈H₈₈N₄O₂₂ [M+H]+m/z 516.3, found 516.3.

Compound 12: Compound 10 (MW: 1072.6, 143 mg, 0.1355 mmol, 1.1 eq) was taken in 50 ml round bottom flask, dissolved in 2 ml THF, then 63.5 mg of Compound 11 (0.1231 mmol) dissolved in THF (2 mL) was added along with CuSO₄·5H2O (6.8 mg, 0.027 mmol, 20 mol %) and sodium ascorbate (10.7 mg, 0.0542 mmol, 40 mol %, and degassed THF: H2O (2 mL, 1:1). The reaction mixture was stirred at 23° C. for 16 h. Next day, the reaction was deemed completed by using thin layer chromatography. The reaction mixture was purified via flash chromatography on 40 g silica column with gradient elution from 100% CH2C12 (mobile phase a) to 75:22:3 CH2C12/MeOH/NH₄OHaq (by volume, mobile phase b). The desired product eluted at 40% mobile phase b. (R_{f}=0.5 in 2:1 mobile phase b/mobile phase a) to yield the compound 12 as white oil (44 mg, 20%). MS (ESI) calculated for C71H135N11O28 [M+H2]²⁺ m/z 794.9, found 795.0.

Compound 13: Compound 12 (MW: 1587.9, 79 mg, 0.05 mmol) was dissolved in 1.25 mL anhydrous DCM, cooled to 0° C. and 0.125 mL of trifluoroacetic acid (TFA) was added. The reaction was stirred at 23° C. for 2 h, evaporated to dryness and dissolved in 0.5 ml DMF, added 0.07 ml Et₃N (0.5 mmol, 10 eq) followed by 80 mg of Ricinoleic-NHS (80 mg, 0.20 mmol, 4 eq.) dissolved in 1 ml DMF. The reaction mixture was stirred at 23° C. for 24 h, and the reaction mixture was purified via flash chromatography on silica column (12 g) with gradient elution from methylene chloride (mobile phase a) to 75:24:6 methylene chloride/methanol/ammonium hydroxide (by volume, mobile phase b). The desired product eluted at 48% mobile phase b. (R_{f}=0.75 in mobile phase b) to yield the desired product as yellow oil (40 mg, 40% over two steps). MS (ESI) calculated for C₉₇H183N11O28 [M+2H]²⁺ m/z 974.2, found 975.3.

Compound 14: Compound 12 (MW: 1587.9, 62 mg, 0.04 mmol) was dissolved in 1 mL anhydrous DCM, cooled to 0° C. and 0.1 mL of trifluoroacetic acid (TFA) was added. The reaction was stirred at 23° C. for 2 h, evaporated to dryness and dissolved in 0.5 ml DMF, added 0.1 ml Et₃N (0.78 mmol, 20 eq) followed by 0.05 ml of octyl acrylate (0.23 mmol, 6 eq, MW 184.3 g/mol, d 0.88 g/ml). The reaction mixture was stirred at 25° C. for 24 h, and the reaction mixture was purified via flash chromatography on silica column (24 g) with gradient elution from 100% CH2C12 (mobile phase a) to 75:22:3 CH2C12/MeOH/NH₄OHaq (by volume, mobile phase b). The desired tetra-octylated product eluted at 48% mobile phase b (R_{f}=0.2 in 1:2 mobile phase a/mobile phase b) to yield yellow oil (10 mg, 12% over two steps). MS (ESI) calculated for C105H198N11O32 [M+2H]²⁺ m/z 1064.2, found 1063.6. 30 mg (40% over two steps) of tri-octylated product was also obtained. MS (ESI) calculated for C94H178N11030 [M+2H]²⁺ m/z 971.35, found 971.4.

Still referring to FIG. 9, nanoparticles were formulated by mixing 30 μl of an ethanol phase containing PEGylated dendrimer (Compound 14) and 14:0 PEG 2000 PE or DMG PEG 2000 (PEG-lipid, Avanti Polar Lipids) with 90 μl of Replicon RNA encoding secreted embryonic alkaline phosphatase (SEAP) diluted with ultraPure, DNase/RNase-free, endotoxin-free distilled water (Invitrogen) and sterile 100 mM (pH 5) citrate buffer to a final citrate concentration of 10 mM. The resulting nanoparticles contained 3:1 or 5:1 mass ratio of nucleic acid carrier to RNA. Formulations were diluted 500-fold for analysis of particle size distribution, Z-average, and derived count rate using a Zetasizer Nano ZS (Malvern Panalytical).

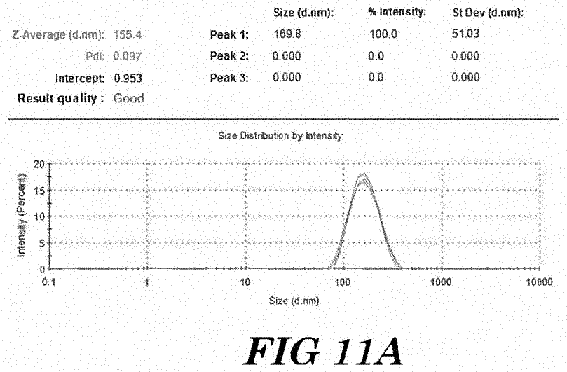

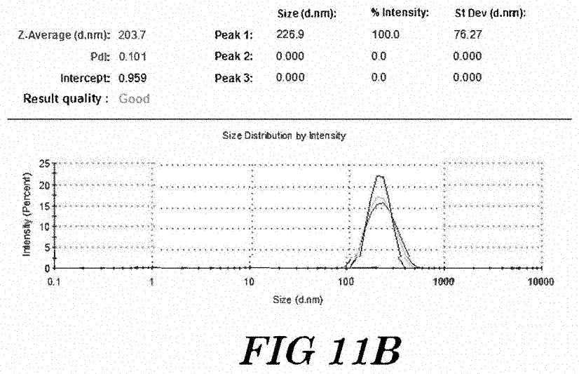

Now referring to FIG. 11, a distribution of the nanoparticle composition measured as the random change in intensity of light (Z average) based on size (d.nm; diameter in nm) of the nanoparticles is shown. The “Z average” of the nanoparticle composition containing Compound 14 and SEAP Replicon RNA (3:1 mass ratio) as a function of size were determined by dynamic light scattering (DLS). Referring to FIGS. 11A and 11B, the strongest intensity was observed for the nanoparticles of 169.8 d.nm in size (14:0 PEG 2000 PE) and 226.9 d.nm (DMG PEG 2000) in size respectively. The size distributions were characterized by a single peak with a low polydispersity index, indicating a relatively monodisperse size (PDI 0.097 and 0.101 respectively).

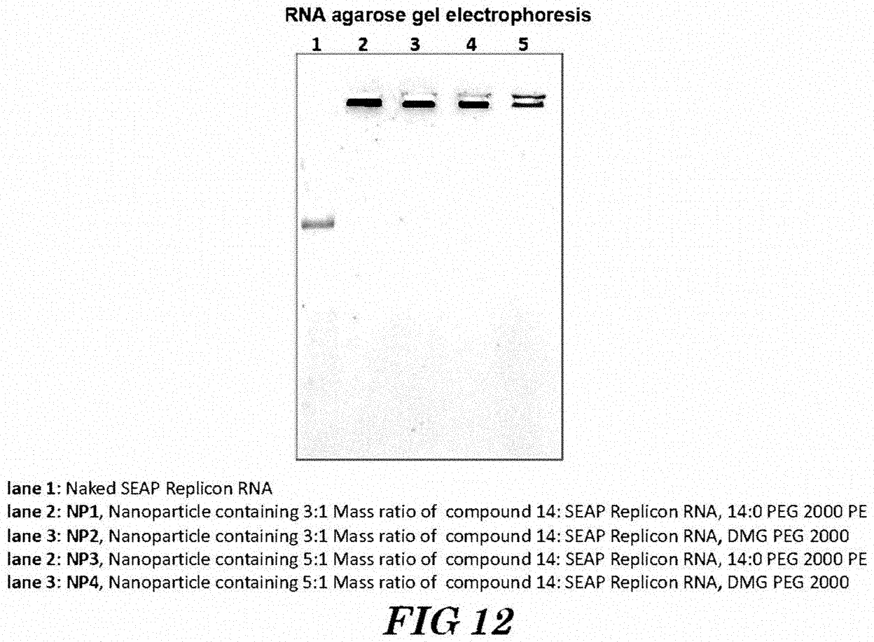

Now referring to FIG. 12, exemplary measurements obtained from gel well-retention assay are shown with a photograph of the agarose gel showing the binding of the PEGylated dendrimer with RNA. Known methods, such as Geall et al. 10.1073/pnas.1209367109 were used. The gels were stained with ethidium bromide (EB) and gel images were taken on a Syngene G Box imaging system (Syngene, USA). Lane 1 contained the unformulated SEAP replicon RNA, lane 2 contained the product of formulation of the Compound 14 and SEAP replicon RNA. Before loading, the samples were incubated with formaldehyde loading dye, denatured for 10 min at 65° C., and cooled to room temperature. The gel was run at 90 V and gel images were taken on a Syngene G Box imaging system (Syngene, USA). The lower band corresponds to the small size free RNA (lane 1) and the top bands (lane 2, lane 3, lane 4 and lane 5) represent the nanoparticles formed by binding of the RNA to the PEGylated dendrimer carrier.

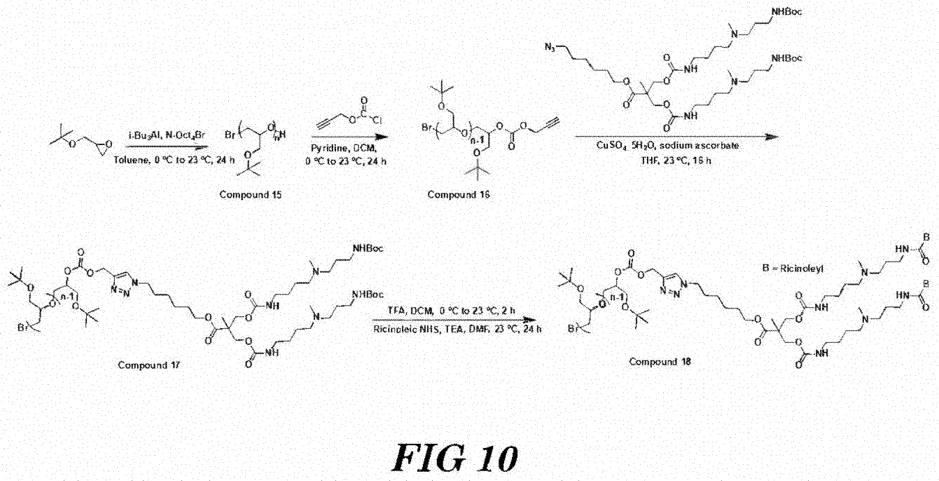

Now referring to FIG. 10, an exemplary synthetic scheme of a Polyglycerol-dendron conjugate as shown in FIG. 10 is a schematic drawing of the synthesis of dendritic polymer conjugate with fatty acid side chains that can be used for helping with self-assembly. In this figure, the fatty acid side chain can be selected from any one of C₄-C₂₈ fatty acids.

Compound 15: The procedure was adapted from literature (Gervais et al, Macromolecules 2010, 43, 4, 1778). Tetraoctylammonium bromide (381.2 mg, 0.7 mmol, 0.1 eq) was taken in a vial equipped with activated molecular sieves, which was then evacuated and re-filled with Argon (once) and dissolved in dry toluene (3.5 mL). Then, tert butyl glycidyl ether (1 mL, 0.7 mmol) was added, the solution was cooled down to 0° C. followed by the dropwise addition of triisobutylaluminum in dry hexane (C=1 M, 1.34 mL, 1.39 mmol, 20 eq). The reaction proceeded for 3 hours at 0° C., at which point, TLC showed complete consumption of the monomer. The reaction was then quenched with ethanol (˜1 mL), solvent was evaporated and the compound was re-dissolved in 30 mL ethyl acetate. MilliQ water (˜1 mL) was then added followed by the addition of Na2SO4. The organic layer was then filtered and then the solvent was removed under reduced pressure and the crude polymer was purified by flash column chromatography on silica column (40 g) with gradient elution from 100% hexanes to (mobile phase a) to 1:1 ethyl acetate (mobile phase b): hexanes (by volume) over 53 minutes. The desired product (Compound 15, Rf=0.7 in 1:1 ethyl acetate:hexanes) eluted at 45% ethyl acetate as clear oil (800 mg, 80%; 1H NMR of the product matched with reported NMR (Gervais et al, Macromolecules 2010, 43, 4, 1778). The Matrix Assisted Laser Desorption/Ionization time-of-flight (MALDI-TOF) of the product showed a molecular weight range (MW=1.01 kDa−2.05 kDa, corresponding to polyglycerol polymers with degrees of polymerization between 7-15) in good agreement with the theoretical mass of the sodium adduct (Theoretical MW=1.3 kDa for degree of polymerization=10) as well as the expected spacings of 130 amu between the species of different degrees of polymerization. 1H NMR of polymer (—O—CH2(1)-CH(2)[CH2(3)-O—[CH3-(4))]3]: 1H NMR (301 MHz, CHLOROFORM-D) 1, 2, 3, 3.20-3.76 ppm (5 H); 4, 1.16 (9 H) ppm.

Compound 16: T-butyl polyglycidol (200 mg, 0.11 mmol) was dissolved in dry DCM (3 mL) and pyridine (89.4 uL, 1.11 mmol, 10 eq) was added followed by propargyl chloroformate (48 uL, 0.55 mmol, 5 eq, MW=118.52), the reaction mixture was stirred at 0° C. to 23° C. for 16 hours. Next day, the reaction mixture was diluted with 1.33 M NaHSO4 and extracted with DCM. The organic layer was washed with brine, concentrated using a rotoevaporator, and purified by flash chromatography with hexanes/ethyl acetate. The compound 16 eluted at 36% ethyl acetate, (Rf=0.7 in 1:1 ethyl acetate/hexane) as clear oil (198 mg, 99%). Presence of propargyl group on the polymer was confirmed by 1H NMR. 1H NMR of the polymer (—O—CH2(1)-CH(2)[CH2(3)-O—[CH3-(4))]3,CH2(5)-C≡CH(6)]: 1H NMR (301 MHz, CHLOROFORM-D) 1, 2, 3, 3.22-3.91 ppm (47 H); 4, 1.15 ppm (88 H); 5, 4.70 ppm (2 H); 6, 4.87 ppm (1 H). Average degree of polymerization (based on number of protons 4) is 10.

Compound 17: To a 25 ml round bottom flask, CuSO₄·5H2O (3.3 mg, 0.013 mmol, 20 mol %, MW 249.69) and sodium ascorbate (5.2 mg, 0.026 mmol, 40 mol %, MW 198.11) were taken. Azide, Compound 4 dissolved in 0.6 ml tetrahydrofuran, then Alkyne, Compound 16 (164.7 mg, 0.066 mmol) dissolved in THF (0.6 mL) was added along with degassed THF: H2O (2 mL, 1:1). The reaction mixture was stirred at 23° C. for 16 h. Next day, thin layer chromatography was used to confirm completion of the reaction. The reaction mixture was purified via flash chromatography on 24 g silica column with gradient elution from 100% methylene chloride (mobile phase a) to 75:24:6 methylene chloride/methanol/ammonium hydroxide (by volume, mobile phase b). The desired product eluted at 80% mobile phase b (Rf=0.48 in 1:2 mobile phase a/mobile phase b) to yield the compound 16 as yellow oil (42 mg, 20%). The successful synthesis was confirmed by the presence of triazole peak observed at 7.64 ppm and disappearance of alkyne CH peak at 4.87 ppm in 1H NMR spectroscopy.

Compound 18: 42 mg of compound 17 (0.013 mmol) was treated with 150 eq of trifluoroacetic acid (0.15 ml, 1.95 mmol) after dissolving the compound in 1.5 ml DCM, the reaction was stirred at 0° C. to 23° C. for 2 h, evaporated to dryness. 1H NMR of the product confirms successful boc deprotection by the disappearance of peak at 1.40 ppm. Next, the compound was dissolved in 0.5 ml DMF, then 0.036 ml Et₃N (0.26 mmol, 20 eq) was added, followed by 41 mg of ricinoleic acid NHS ester (0.052 mmol, 8 eq). The reaction mixture was stirred at 23° C. for 24 h, and then was purified via flash chromatography on silica column (24 g) with gradient elution from 100% CH2C12 (mobile phase a) to 75:22:3 CH2C12/MeOH/NH4OHaq (by volume, mobile phase b). The desired product eluted at 46% mobile phase b (Rf=0.7 in 1:2 mobile phase a/mobile phase b) as yellow oil (7 mg, 20% over two steps). The MALDI-TOF of product showed a molecular weight range in good agreement with the theoretical mass of the potassium adduct (MW=1.89 kDa-2.69 kDa, corresponding to dendron conjugated polyglycerol polymers with degree of polymerization between 5-10) as well as the expected spacings of 130 atomic mass units between the species of different degrees of polymerization.

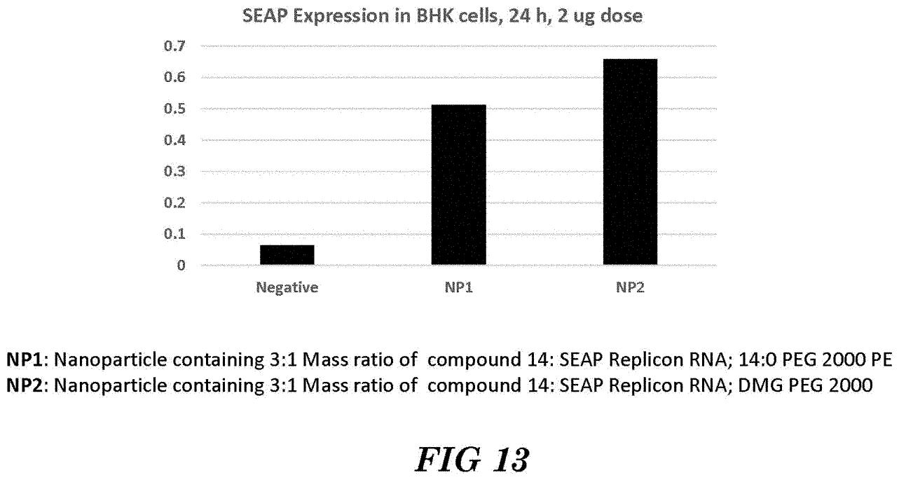

Now referring to FIG. 13, optical density measurements showing the expression of secreted alkaline phosphatase (SEAP) with nanoparticle formulations using PEGylated dendrimer, compound 14. To test the ability of the nanoparticles formulated with PEGylated carrier to express SEAP in vitro, BHK cells were treated with nanoparticles. Each well of a 12 well dish of BHKs was treated with 50 μL (approximately 2 μg) of each formulation product diluted into a final volume of 500 μL with a 1:1 Optimem:PBS mix. After the treatment, BHK cells were incubated at 37° C. and 5% CO2. After 24 hours, cell culture medium was collected and assayed for SEAP using the InvivoGen QUANTI-Blue™ detection system (San Diego, CA, USA), according to the manufacturer's protocol. Briefly, 50 μL of the cell culture medium was added to 150 μL of the QUANTI-Blue™ solution and incubated at 37° C. for 10 minutes. The Optical Density (OD) was measured at 620-655 nm using a microplate reader. FIG. 13 illustrates the SEAP expression of nanoparticle formulations using compound 14 based on optical density compared to the negative control. Formulation with DMG PEG 2000 had higher expression than that with 14:0 PEG 2000 in vitro at 24 h.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, compositions, and desired results according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A nanoparticle composition for delivery of a nucleic acid agent, comprising: a nucleic acid agent and a carrier, wherein the carrier comprises:
a dendritic polymer skeleton;
an amine; and
a hydrophobic unit, wherein:
the dendritic polymer skeleton comprises a PEG moiety or PEG alternatives;

2. The nanoparticle composition of claim 1, wherein the nucleic acid agent is composed at least in part of at least one type of ribonucleic acid.

3. The nanoparticle composition of claim 1, wherein the nucleic acid agent is composed at least in part of at least one type of deoxyribonucleic acid.

4. The nanoparticle composition of claim 1, wherein the amine comprises a secondary amine.

5. The nanoparticle composition of claim 1, wherein the amine comprises a tertiary amine.

6. The nanoparticle composition of claim 1, wherein the hydrophobic unit comprises an alkyl chain.

7. The nanoparticle composition of claim 6, wherein the alkyl chain includes a number of carbon atoms ranging from 1 carbon atom to 28 carbon atoms.

8. The nanoparticle composition of claim 1, wherein the hydrophobic unit comprises an alkenyl chain.

9. The nanoparticle composition of claim 8, wherein the alkenyl chain includes a number of carbon atoms ranging from 2 carbon atom to 28 carbon atoms.

10. The nanoparticle composition of claim 1, wherein the PEG moiety has a weight of about 100 Daltons to about 750 Daltons.

11. The nanoparticle composition of claim 1, wherein the PEG moiety has a length of 1 monomer to 15 monomers.

12. The nanoparticle composition of claim 1, wherein the carrier further comprises a functional group suitable for tracking the carrier.

13. The nanoparticle composition of claim 1, further comprising a polymer-lipid conjugate.

14. The nanoparticle composition of claim 13, wherein the polymer-lipid conjugate is a PEG-lipid conjugate.

15. The nanoparticle composition of claim 14, wherein the PEG-lipid conjugate is 1,2-dimyristoyl-snglycero-3-phosphoethanolamine-N-[methoxy (poly-ethylene glycol)-2000] or 1,2-dimyristoyl-rac-glycero-3-methoxypolyethylene glycol-2000.

16. The nanoparticle composition of claim 14, wherein the PEG-lipid conjugate is present in a concentration ranging from about 1 mol % to about 10 mol %.

17. The nanoparticle composition of claim 1, further comprising an amphipathic lipid.

18. The nanoparticle composition of claim 17, wherein the amphipathic lipid is a phospholipid.

19. The nanoparticle composition of claim 18, wherein the phospholipid is 1,2-dioleoyl-snglycero-3-phosphoethanolamine (DOPE) or distearoylphosphatidylcholine (DSPC).

20. The nanoparticle composition of claim 18, wherein the phospholipid is present in a concentration ranging from about 10 mol % to about 25 mol %.

21. The nanoparticle composition of claim 1, further comprising cholesterol or a derivative thereof.

22. The nanoparticle composition of claim 21, wherein the cholesterol or derivative thereof is present in a concentration ranging from about 50 mol % to about 75 mol %.

23. A method of managing disease, the method comprising:
administering a therapeutically effective amount of a nanoparticle composition to a subject,
wherein the nanoparticle composition comprises: a nucleic acid agent and a nucleic acid carrier, wherein the carrier comprises:
a dendritic-polymer skeleton;
an amine; and
a hydrophobic unit, wherein:
the dendritic-polymer skeleton comprises a PEG moiety; and
the amine connects the hydrophobic unit to the dendritic-polymer skeleton.

24. The method of claim 23, wherein the PEG moiety has a length of 1 monomer to 15 monomers.

25. The method of claim 23, wherein the nucleic acid agent comprises a ribonucleic acid.

26. The method of claim 23, wherein the nucleic acid agent comprises a deoxyribonucleic acid.

27. The method of claim 23, wherein the nucleic acid agent is present in a concentration in the range of about 0.01 mg to about 10 mg of nucleic acid agent per kg body weight of the subject.
